(19) United States
(12) Patent Application Publication
(10) Pub. No.: US 2026/0256445 A1
(43) Pub. Date: Sep. 3, 2026

(54) ULTRASONIC DIAGNOSTIC APPARATUS, METHOD, AND RECORDING MEDIUM

(71) Applicant: CANON MEDICAL SYSTEMS CORPORATION, Tochigi (JP)

(72) Inventors: Kuramitsu NISHIHARA, Otawara-shi (JP); Chihiro SHIBATA, Nasushiobara-shi (JP); Akihiro KAKEE, Nasushiobara-shi (JP)

(73) Assignee: CANON MEDICAL SYSTEMS CORPORATION, Tochigi (JP)

(21) Appl. No.: 19/550,525

(22) Filed: Feb. 26, 2026

(30) Foreign Application Priority Data
Feb. 28, 2025 (JP) 2025-032163

(51) Int. Cl. A61B 8/06; A61B 8/00

(52) U.S. Cl. CPC A61B 8/06; A61B 8/463; A61B 8/467; A61B 8/488; A61B 8/5207; A61B 8/5223; A61B 8/5246; A61B 8/543

(57) ABSTRACT

An ultrasonic diagnostic apparatus of an embodiment generates a color Doppler image using a plurality of pieces of transmission/reception data obtained by performing ultrasonic transmission/reception multiple times on the same scanning line. The ultrasonic diagnostic apparatus includes processing circuitry. The processing circuitry collects a plurality of pieces of transmission/reception data by multiple ultrasonic transmissions/transmissions on a predetermined scanning line. The processing circuitry determines a number of pieces of transmission/reception data not to be used for color Doppler image generation according to an effect of an echo signal transmitted/received at a time phase prior to the multiple ultrasonic transmissions/receptions on the predetermined scanning line, and generates a color Doppler image using a plurality of pieces of transmission/reception data, excluding a piece or pieces of transmission/reception data corresponding to the determined number of pieces of transmission/reception data counted from a first piece of transmission/reception data, among the collected pieces of transmission/reception data.

### CROSS-REFERENCE TO RELATED APPLICATIONS

This application is based upon and claims the benefit of priority from Japanese Patent Application No. 2025-032163, filed on Feb. 28, 2025; the entire contents of which are incorporated herein by reference.

### FIELD

Embodiments described herein relate generally to an ultrasonic diagnostic apparatus, a method, and a recording medium.

### BACKGROUND

There is an ultrasonic diagnostic apparatus that performs the transmission and reception of ultrasonic waves to and from a subject to image the internal conditions of the subject. In such an ultrasonic diagnostic apparatus, various modes are set to generate various types of image data.

When ultrasonic waves are transmitted and received in an electronic scan in the ultrasonic diagnostic apparatus, if the signal intensity on a scanning line immediately before switching positions is relatively strong, signals that have not fully attenuated may be superimposed (be received) as residual signals on the scanning line after switching positions (current scanning line). This is called residual multiplex.

Such residual multiplex may occur, for example, in a color Doppler mode (blood flow display mode) that generates color Doppler image data visualizing blood flow in a subject and displays a color Doppler image based on the color Doppler image data on a display. When the ultrasonic diagnostic apparatus performs phase detection (phase sensitive detection), which is performed in the color Doppler mode, using signals affected by residual multiplex, the color Doppler image displayed on the display may contain noise such as stripes as artifacts. This is because the correlation between a plurality of signals is relatively weak. When residual multiplex occurs, the position of scan by the ultrasound probe is changed to reduce residual multiplex. This may lead to lower inspection efficiency.

Another technique to reduce residual multiplex is dummy rate transmission. For example, the dummy rate transmission is a technique that performs transmission and reception of ultrasonic waves for a scanning line immediately before switching positions, then waits for a specified time, and after the specified time has elapsed, starts transmission and reception of ultrasonic waves for the current scanning line. By waiting for a specified time, the intensity of residual signals is sufficiently reduced. As a result, different residual signals do not enter each of a plurality of scanning lines. In other words, similar residual signals enter each of a plurality of scanning lines. This reduces residual multiplex.

However, in dummy rate transmission, the frame rate is reduced and real-time performance may be compromised because of a wait for a specified time after the transmission and reception of ultrasonic waves for a scanning line immediately before switching positions.

### BRIEF DESCRIPTION OF THE DRAWINGS

FIG. 1 is a block diagram illustrating an example configuration of an ultrasonic diagnostic apparatus according to a first embodiment;

FIG. 2 is a diagram for explaining an example of first ultrasonic scanning and second ultrasonic scanning in a normal mode according to the first embodiment;

FIG. 3 is a diagram illustrating an example of the positions of a plurality of scanning lines (positions in spatial coordinates of image data) in each scan of the first ultrasonic scanning and the second ultrasonic scanning according to the first embodiment;

FIG. 4 is a diagram for explaining dummy rate transmission executed by an ultrasonic diagnostic apparatus according to a comparative example when a dummy rate mode is set as a scanning mode;

FIG. 5 is a diagram illustrating an example of a data sequence input to an eigenvector MTI filter in the normal mode illustrated in FIG. 2;

FIG. 6 is a diagram illustrating an example of a data sequence input to the eigenvector MTI filter in a residual multiplex reduction mode according to the first embodiment;

FIG. 7 is a flowchart illustrating the flow of exemplary processing executed by the ultrasonic diagnostic apparatus according to the first embodiment;

FIG. 8 is a diagram illustrating an example of a color Doppler image displayed by the ultrasonic diagnostic apparatus according to the first embodiment;

FIG. 9 is a block diagram illustrating an example configuration of an ultrasonic diagnostic apparatus according to a second embodiment;

FIG. 10 is a diagram for explaining an example of first ultrasonic scanning and second ultrasonic scanning in the normal mode according to the second embodiment;

FIG. 11 is a diagram illustrating an example of a data sequence input to the eigenvector MTI filter in the residual multiplex reduction mode according to the second embodiment;

FIG. 12 is a diagram for explaining an example of first ultrasonic scanning and second ultrasonic scanning in the dummy rate mode according to the second embodiment;

FIG. 13 is a flowchart illustrating the flow of exemplary processing executed by the ultrasonic diagnostic apparatus according to the second embodiment;

FIG. 14 is a diagram illustrating an example of a color Doppler image displayed by the ultrasonic diagnostic apparatus according to the second embodiment;

FIG. 15 is a diagram for explaining the features of color Doppler images generated in the ultrasonic diagnostic apparatus according to the second embodiment, for each combination of level and scanning mode;

FIG. 16 is a block diagram illustrating an example configuration of an ultrasonic diagnostic apparatus according to a third embodiment;

FIG. 17 is a diagram for explaining an example of processing for detecting residual multiplex in the third embodiment;

FIG. 18 is a diagram for explaining an example of processing for detecting whether there is pulsatility in the third embodiment;

FIG. 19 is a diagram for explaining an example of processing for detecting whether there is pulsatility in the third embodiment;

FIG. 20 is a flowchart illustrating the flow of exemplary processing executed by the ultrasonic diagnostic apparatus according to the third embodiment;

FIG. 21 is a diagram for explaining an example of the first ultrasonic scanning in a mode division scan; and

FIG. 22 is a diagram illustrating an example of the positions of a plurality of scanning lines (positions in spatial coordinates of image data) in the first ultrasonic scanning in the mode division scan.

### DETAILED DESCRIPTION

One of the problems to be solved by the embodiments disclosed herein and the drawings is to reduce the effect of residual multiplex on color Doppler image data while suppressing the reduction in the frame rate of color Doppler image data. However, the problems to be solved by the embodiments disclosed herein and in the drawings are not limited to the above problem. Problems corresponding to the effects achieved by the configurations illustrated in the embodiments described below can also be considered as other problems.

An ultrasonic diagnostic apparatus of an embodiment generates a color Doppler image using a plurality of pieces of transmission/reception data obtained by performing ultrasonic transmission/reception multiple times on the same scanning line. The ultrasonic diagnostic apparatus includes processing circuitry. The processing circuitry collects a plurality of pieces of transmission / reception data by multiple ultrasonic transmissions / receptions on a predetermined scanning line. The processing circuitry determines a number of pieces of transmission/reception data not to be used for color Doppler image generation according to an effect of an echo signal transmitted/received at a time phase prior to the multiple ultrasonic transmissions / receptions on the predetermined scanning line, and generates a color Doppler image using a plurality of pieces of transmission / reception data, excluding a piece or pieces of transmission / reception data corresponding to the determined number of pieces of transmission / reception data counted from a first piece of transmission/reception data, among the pieces of transmission/reception data collected by the collection unit.

An ultrasonic diagnostic apparatus, a method, and a computer program according to each of embodiments will be described below with reference to the drawings. Hereinafter, parts denoted with the same reference signs are assumed to operate in the same way, and duplicated descriptions may be omitted as appropriate. The embodiments can be combined with other embodiments or conventional technologies to the extent that there is no inconsistency in the contents of processing.

### First Embodiment

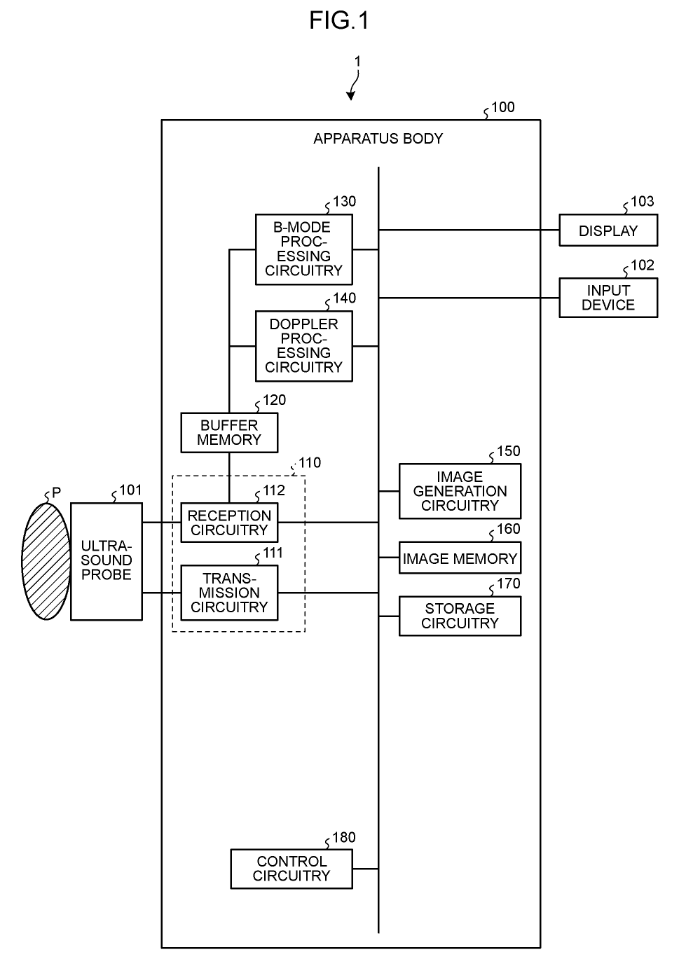

FIG. 1 is a block diagram illustrating an example configuration of an ultrasonic diagnostic apparatus 1 according to a first embodiment. As illustrated in FIG. 1, the ultrasonic diagnostic apparatus 1 according to the first embodiment includes an apparatus body 100, an ultrasound probe 101, an input device 102, and a display 103.

The ultrasound probe 101 has, for example, a plurality of elements (piezoelectric transducer elements, piezoelectric elements). These elements generate ultrasonic waves based on drive signals supplied by transmission circuitry 111 of transmission/reception circuitry 110 of the apparatus body 100. Specifically, the elements generate an ultrasonic wave having a waveform corresponding to a transmission drive voltage when a voltage (transmission drive voltage) is applied by the transmission circuitry 111. The waveform of the transmission drive voltage indicated by the drive signal is the waveform of the voltage applied to the elements. In other words, the ultrasound probe 101 transmits an ultrasonic wave according to the magnitude of the applied transmission drive voltage. The ultrasound probe 101 receives a reflected wave from a subject P, converts the received reflected wave into a reflected wave signal, which is an electrical signal, and outputs the reflected wave signal to the apparatus body 100. The ultrasound probe 101 has, for example, a matching layer on the elements and a backing material that prevents the propagation of ultrasonic waves from the elements to the back. The ultrasound probe 101 is detachably connected to the apparatus body 100.

When ultrasonic waves are transmitted from the ultrasound probe 101 to the subject P, the transmitted ultrasonic waves are reflected one after another at the acoustic impedance discontinuous surface in the body tissue of the subject P, and are received as reflected waves by the elements of the ultrasound probe 101. The amplitude of the received reflected waves depends on the difference in acoustic impedance at the discontinuous surface at which ultrasonic waves are reflected. When the transmitted ultrasonic pulse is reflected at a surface of a moving object, such as moving blood flow or heart wall, the reflected wave undergoes a frequency shift depending on a velocity component with respect to the direction of ultrasonic transmission of the moving object due to the Doppler effect. The ultrasound probe 101 then outputs the reflected wave signal to reception circuitry 112 of the transmission/reception circuitry 110 described below.

The ultrasound probe 101 is detachable from the apparatus body 100. When a two-dimensional region within the subject P is scanned (two-dimensional scanning), the operator connects, for example, a 1D array probe with a plurality of elements in a row as the ultrasound probe 101 to the apparatus body 100. Types of the 1D array probe include linear, convex, and sector ultrasound probes. When a three-dimensional region within the subject P is scanned (three-dimensional scanning), the operator connects, for example, a mechanical 4D probe or 2D array probe as the ultrasound probe 101 to the apparatus body 100. The mechanical 4D probe is capable of two-dimensional scanning using a plurality of elements arranged in a row, like a 1D array probe, and capable of three-dimensional scanning by swinging a plurality of elements at a predetermined angle (swing angle). The 2D array probe is capable of three-dimensional scanning with a plurality of elements arranged in a matrix and capable of two-dimensional scanning by focusing and transmitting ultrasonic waves.

The input device 102 is implemented, for example, by input means such as a mouse, a keyboard, buttons, panel switches, a touch command screen, a foot switch, a trackball, and a joystick. The input device 102 accepts various setting requests from the operator of the ultrasonic diagnostic apparatus 1 and transfers the accepted setting requests to the apparatus body 100. Here, the input device 102 according to the present embodiment includes a residual multiplex reduction button (not illustrated). For example, the operator presses the residual multiplex reduction button to reduce the effect of residual multiplex on color Doppler image data while suppressing the reduction in the frame rate of the color Doppler image data.

The display 103, for example, displays a graphical user interface (GUI) for the operator of the ultrasonic diagnostic apparatus 1 to input various setting requests using the input device 102, or displays an ultrasonic image based on ultrasonic image data generated in the apparatus body 100. The display 103 is implemented by a liquid crystal monitor, an organic light emitting diode (OLED) monitor, or the like. The display 103 is an example of a display unit.

The apparatus body 100 generates ultrasonic image data based on reflected wave signals transmitted from the ultrasound probe 101. The ultrasonic image data is an example of image data. The apparatus body 100 can generate two-dimensional ultrasonic image data based on reflected wave signals corresponding to a two-dimensional region of the subject P transmitted from the ultrasound probe 101. The apparatus body 100 can generate three-dimensional ultrasonic image data based on reflected wave signals corresponding to a three-dimensional region of the subject P transmitted from the ultrasound probe 101. As illustrated in FIG. 1, the apparatus body 100 includes transmission/reception circuitry 110, a buffer memory 120, B-mode processing circuitry 130, Doppler processing circuitry 140, image generation circuitry 150, an image memory 160, storage circuitry 170, and control circuitry 180.

The transmission/reception circuitry 110 allows the ultrasound probe 101 to transmit ultrasonic waves and allows the ultrasound probe 101 to receive reflected waves (echoes) of the ultrasonic waves, under control by the control circuitry 180. In other words, the transmission/reception circuitry 110 executes scanning through the ultrasound probe 101. As used herein, for example, "transmission/reception" means collecting data on a single scanning line by transmitting and receiving ultrasonic waves (by transmission and reception of ultrasonic waves), and "scanning" means repetition of "transmission/reception". Transmission and reception of ultrasonic waves is also referred to simply as ultrasonic transmission/reception. Transmitting and receiving ultrasonic waves means, for example, transmitting an ultrasonic wave and receiving the reflected wave of the transmitted ultrasonic wave. "Scanning" is also referred to as ultrasonic scanning. For example, "scan" means both "transmission/reception" and "scanning". The transmission/reception circuitry 110 is an example of a transmitter/receiver. The transmission/reception circuitry 110 includes the transmission circuitry 111 and the reception circuitry 112. The transmission circuitry 111 is an example of a transmitter, and the reception circuitry 112 is an example of a receiver.

The transmission circuitry 111 supplies a drive signal to the ultrasound probe 101 under control by the control circuitry 180 to allow the ultrasound probe 101 to transmit an ultrasonic wave. The transmission circuitry 111 has a rate pulser generation circuit, a transmission delay circuit, and a transmission pulser. When a two-dimensional region within the subject P is scanned, the transmission circuitry 111 allows the ultrasound probe 101 to transmit an ultrasound beam for scanning the two-dimensional region. When a three-dimensional region within the subject P is scanned, the transmission circuitry 111 allows the ultrasound probe 101 to transmit an ultrasound beam for scanning the three-dimensional region.

The rate pulser generation circuit repeatedly generates a rate pulse for forming a transmission ultrasonic wave (transmission beam) at a predetermined pulse repetition frequency (PRF) under control by the control circuitry 180. As the rate pulse passes through the transmission delay circuit, a voltage is applied to the transmission pulser with different transmission delay times. For example, the transmission delay circuit applies, to each rate pulse generated by the rate pulser generation circuit, a transmission delay time for each element that is necessary to focus the ultrasonic waves generated by the ultrasound probe 101 into a beam and determine the transmission directivity. The transmission pulser supplies a drive signal (drive pulse) to the ultrasound probe 101 at a timing based on the rate pulse. In other words, the transmission pulser applies a voltage with a waveform indicated by the drive signal (transmission drive voltage) to the ultrasound probe 101 at the timing based on the rate pulse. The transmission delay circuit adjusts the transmission direction of ultrasonic waves from the element surface as desired by varying the transmission delay time applied to each rate pulse.

The drive pulse is transmitted from the transmission pulser to the element in the ultrasound probe 101 via a cable, and then converted from an electrical signal to mechanical vibration in the element. In other words, the element vibrates mechanically when a voltage is applied to the element. The ultrasonic wave generated by this mechanical vibration is transmitted inside the living body (inside the subject P). Here, ultrasonic waves with different transmission delay times for each element are focused and propagate in a predetermined direction.

The transmission circuitry 111 has a function capable of instantaneously changing a transmission frequency, a transmission drive voltage, and the like to execute a predetermined scanning sequence, under control by the control circuitry 180. In particular, the changing of a transmission drive voltage is realized by a linear amplifier type transmission circuitry that can instantaneously switch the value of the transmission drive voltage, or by a mechanism that electrically switches a plurality of power supply units. The transmission frequency is, for example, the center frequency of the transmitted ultrasonic wave.

The reflected wave of the ultrasonic wave transmitted by the ultrasound probe 101 reaches the element inside the ultrasound probe 101 and is then converted from mechanical vibration to an electrical signal (reflected wave signal) in the element, and the reflected wave signal is input to the reception circuitry 112. The reception circuitry 112 includes a preamplifier, an analog to digital (A/D) converter, a quadrature detection circuit, and the like, and performs various processing on the reflected wave signal transmitted from the ultrasound probe 101 to generate reflected wave data. The reception circuitry 112 then stores the generated reflected wave data into the buffer memory 120.

The preamplifier amplifies the reflected wave signal for each channel and performs gain adjustment (gain correction). The A/D converter converts the gain-corrected reflected wave signal to a digital signal by A/D conversion of the gain-corrected reflected wave signal. The quadrature detection circuit converts the reflected wave signal converted to a digital signal into an in-phase signal (I signal, I: In-phase) and a quadrature signal (Q signal, Q: Quadrature-phase) in the baseband band. The quadrature detection circuit then stores the I and Q signals (IQ signals) as reflected wave data into the buffer memory 120.

The reception circuitry 112 performs various processing on the reflected wave signal transmitted from the ultrasound probe 101 to generate reflected wave data. The reception circuitry 112 then stores the generated reflected wave data into the buffer memory 120.

The reception circuitry 112 generates two-dimensional reflected wave data from a two-dimensional reflected wave signal transmitted from the ultrasound probe 101. The reception circuitry 112 also generates three-dimensional reflected wave data from a three-dimensional reflected wave signal transmitted from the ultrasound probe 101.

In the present embodiment, the ultrasonic diagnostic apparatus 1 can perform various processing in real time. For example, the ultrasound probe 101 transmits the reflected wave signals for one frame one after another to the reception circuitry 112. Each time the reception circuitry 112 receives the reflected wave signals for one frame transmitted from the ultrasound probe 101, the reception circuitry 112 generates reflected wave data for one frame from the reflected wave signals for one frame. Each time the reception circuitry 112 generates reflected wave data for one frame, the reception circuitry 112 stores the reflected wave data for one frame into the buffer memory 120.

The buffer memory 120 is a memory that temporarily stores therein reflected wave data generated by the transmission/reception circuitry 110. For example, the buffer memory 120 is configured to store therein reflected wave data for a predetermined number of frames. When reflected wave data for one frame is newly generated by the reception circuitry 112 while the buffer memory 120 stores therein a predetermined number of frames of reflected wave data, the buffer memory 120 discards the reflected wave data for one frame generated earliest and stores therein the newly generated reflected wave data for one frame, under the control of the reception circuitry 112. For example, the buffer memory 120 is implemented by a semiconductor memory element such as a random access memory (RAM) or a flash memory.

The B-mode processing circuitry 130 reads reflected wave data from the buffer memory 120, performs various signal processing on the read reflected wave data, and outputs the reflected wave data subjected to various signal processing as B-mode data to the image generation circuitry 150. The B-mode processing circuitry 130 is implemented, for example, by a processor. The B-mode processing circuitry 130 is an example of a B-mode processing unit.

For example, each time reflected wave data for one frame is newly stored into the buffer memory 120, the B-mode processing circuitry 130 reads the reflected wave data for one frame newly stored in the buffer memory 120. The B-mode processing circuitry 130 then performs various signal processing on the read reflected wave data for one frame to newly generate B-mode data for one frame. Each time the B-mode processing circuitry 130 generates B-mode data for one frame, the B-mode processing circuitry 130 outputs the newly generated B-mode data for one frame to the image generation circuitry 150. An example of various signal processing executed by the B-mode processing circuitry 130 will be described below.

For example, the B-mode processing circuitry 130 performs quadrature detection, logarithmic amplification and envelope detection processing, and the like on the reflected wave data read from the buffer memory 120 to generate B-mode data representing the signal intensity (amplitude intensity) for each sample point in terms of brightness of luminance. The B-mode processing circuitry 130 then outputs the generated B-mode data to the image generation circuitry 150.

The Doppler processing circuitry 140 reads reflected wave data from the buffer memory 120, performs various signal processing on the read reflected wave data, and outputs the reflected wave data subjected to various signal processing as Doppler data to the image generation circuitry 150. The Doppler processing circuitry 140 is implemented, for example, by a processor. The Doppler processing circuitry 140 is an example of a Doppler processing unit.

For example, each time reflected wave data for one frame is newly stored into the buffer memory 120, the Doppler processing circuitry 140 reads the reflected wave data for one frame newly stored in the buffer memory 120. The Doppler processing circuitry 140 then performs various signal processing on the read reflected wave data for one frame to newly generate Doppler data for one frame. Each time the Doppler processing circuitry 140 generates Doppler data for one frame, the Doppler processing circuitry 140 outputs the newly generated Doppler data for one frame to the image generation circuitry 150. An example of various signal processing executed by the Doppler processing circuitry 140 will be described below.

For example, the Doppler processing circuitry 140 extracts motion information of a moving object (blood flow, tissue, contrast medium echo components, etc.) based on the Doppler effect from the reflected wave data by frequency analysis of the reflected wave data read from the buffer memory 120, and generates Doppler data indicating the extracted motion information. For example, the Doppler processing circuitry 140 extracts average velocity, average variance, average power, and the like over multiple points, as motion information of a moving object, and generates Doppler data indicating the extracted motion information of the moving object. The Doppler processing circuitry 140 outputs the generated Doppler data to the image generation circuitry 150.

Using the functions of the Doppler processing circuitry 140 described above, the ultrasonic diagnostic apparatus 1 can execute a color Doppler method, also called a color flow mapping (CFM) method. In the color flow mapping method, transmission and reception of ultrasonic waves are performed multiple times on each of a plurality of scanning lines. In the color flow mapping method, a moving target indicator (MTI) filter is applied to a data sequence at the same location to suppress a signal originating from stationary or slow-moving tissue (clutter signal) and extract a signal originating from blood flow (blood flow signal) from the data sequence at the same location. In the color flow mapping method, blood flow information such as blood flow velocity (average velocity), blood flow variance (average variance), and blood flow power (average power) is estimated from the blood flow signal. The Doppler processing circuitry 140 outputs color Doppler data indicating blood flow information estimated by the color flow mapping method to the image generation circuitry 150.

The Doppler processing circuitry 140 according to the present embodiment uses, as an MTI filter, an adaptive MTI filter that changes its coefficients according to an input signal. For example, the Doppler processing circuitry 140 uses an adaptive MTI filter called "eigenvector regression filter". The "eigenvector regression filter", which is an adaptive MTI filter using eigenvectors, is hereinafter referred to as "eigenvector MTI filter".

The eigenvector MTI filter calculates eigenvectors from a correlation matrix and calculates, from the calculated eigenvectors, coefficients used in the clutter component suppressing process. This method is an application of the techniques used in principal component analysis, Karhunen-Loeve transform, and the eigenspace method.

The Doppler processing circuitry 140 according to the first embodiment using the eigenvector MTI filter calculates the correlation matrix of a first sub-region described below, from a data sequence of consecutive reflected wave data at the same location (same sample point). The Doppler processing circuitry 140 then calculates the eigenvalues of the correlation matrix and the eigenvectors corresponding to the eigenvalues. The Doppler processing circuitry 140 then calculates, as a filter matrix that suppresses a clutter component, a matrix that reduces the rank of the matrix in which the eigenvectors are arranged based on the magnitude of each eigenvalue.

The Doppler processing circuitry 140 then uses the filter matrix to identify a data sequence from which the clutter component is suppressed and the blood flow signal originating from blood flow is extracted, from the data sequence of consecutive reflected wave data at the same location (same sample point). The Doppler processing circuitry 140 then estimates blood flow information by performing calculations such as autocorrelation operations using the identified data sequence. The Doppler processing circuitry 140 then outputs color Doppler data indicating the estimated blood flow information to the image generation circuitry 150. In this way, the Doppler processing circuitry 140 outputs the color Doppler data to the image generation circuitry 150, for each first sub-region described later.

The B-mode processing circuitry 130 and the Doppler processing circuitry 140 can process both two-dimensional reflected wave data and three-dimensional reflected wave data.

The image generation circuitry 150 generates various ultrasonic image data from B-mode data, second harmonic components, and third harmonic components output from the B-mode processing circuitry 130, and Doppler data and color Doppler data output from the Doppler processing circuitry 140. For example, the image generation circuitry 150 is implemented by a processor.

For example, the image generation circuitry 150 generates two-dimensional B-mode image data representing the intensity of the reflected wave in terms of luminance, from the two-dimensional B-mode data generated by the B-mode processing circuitry 130. The image generation circuitry 150 generates two-dimensional Doppler image data or two-dimensional color Doppler image data visualizing motion information or blood flow information from the two-dimensional Doppler data or color Doppler data generated by the Doppler processing circuitry 140. The two-dimensional Doppler image data visualizing motion information and the two-dimensional color Doppler image data visualizing blood flow information are velocity image data, variance image data, power image data, or image data of a combination of these.

Here, the image generation circuitry 150 generally converts (scan-converts) a scanning line signal sequence of ultrasonic scanning into a video-format scanning line signal sequence as typified by television or the like, and generates ultrasonic image data for display. For example, the image generation circuitry 150 generates ultrasonic image data for display by performing coordinate transformation on data output from the B-mode processing circuitry 130 and the Doppler processing circuitry 140 according to the form of ultrasonic scanning by the ultrasound probe 101. In addition to scan conversion, the image generation circuitry 150 may also perform various image processing, such as image processing to regenerate an average image of luminance using a plurality of image frames after scan conversion (smoothing process) or image processing using a differential filter in an image (edge enhancement process). The image generation circuitry 150 may also combine text information, scales, body marks, and the like for various parameters into the ultrasonic image data.

Furthermore, the image generation circuitry 150 generates three-dimensional B-mode image data by performing coordinate transformation on three-dimensional B-mode data generated by the B-mode processing circuitry 130. The image generation circuitry 150 also generates three-dimensional Doppler image data by performing coordinate transformation on three-dimensional Doppler data generated by the Doppler processing circuitry 140. In other words, the image generation circuitry 150 generates "three-dimensional B-mode image data and three-dimensional Doppler image data" as "three-dimensional ultrasonic image data (volume data)". The image generation circuitry 150 then performs various rendering processes on the volume data to generate various two-dimensional image data for displaying the volume data on the display 103.

The rendering processes performed by the image generation circuitry 150 include, for example, a process of generating MPR image data from the volume data using a multi planer reconstruction (MPR) method. The rendering processes performed by the image generation circuitry 150 include, for example, a volume rendering (VR) process to generate two-dimensional image data reflecting three-dimensional information. The image generation circuitry 150 is an example of an image generation unit.

The B-mode data and the Doppler data are ultrasonic image data before the scanning conversion process, and the data generated by the image generation circuitry 150 is ultrasonic image data for display after the scanning conversion process. The B-mode data and the Doppler data are also referred to as raw data.

The image memory 160 is a memory that stores therein various image data generated by the image generation circuitry 150. The image memory 160 also stores therein data generated by the B-mode processing circuitry 130 and the Doppler processing circuitry 140. The B-mode data and the Doppler data stored in the image memory 160, for example, can be invoked by the operator after diagnosis and become ultrasonic image data for display via the image generation circuitry 150. For example, the image memory 160 is implemented by a semiconductor memory element such as a random access memory (RAM) or a flash memory, a hard disk, or an optical disk.

The storage circuitry 170 stores therein a control program for performing scanning, image processing, and display processing, as well as diagnostic information (e.g., patient ID, doctor's findings, etc.) and various data such as diagnostic protocols and various body marks. The storage circuitry 170 is also used to archive therein data stored in the image memory 160, if necessary. For example, the storage circuitry 170 is implemented by a semiconductor memory element such as a flash memory, a hard disk, or an optical disk.

The control circuitry 180 controls the entire processing of the ultrasonic diagnostic apparatus 1. Specifically, the control circuitry 180 controls the processing in the transmission circuitry 111, the reception circuitry 112, the B-mode processing circuitry 130, the Doppler processing circuitry 140, and the image generation circuitry 150, based on various setting requests input from the operator through the input device 102, and various control programs and various data read from the storage circuitry 170. The control circuitry 180 also controls the display 103 to display ultrasonic images based on ultrasonic image data for display stored in the image memory 160. For example, the control circuitry 180 controls the display 103 to display a B-mode image based on the B-mode image data or a color Doppler image based on the color Doppler image data. The control circuitry 180 also controls the display 103 to superimpose a color Doppler image on a B-mode image.

The control circuitry 180 is an example of a display control unit or a control unit. The control circuitry 180 is implemented, for example, by a processor.

The control circuitry 180 also controls the ultrasonic scanning by controlling the ultrasound probe 101 through the transmission/reception circuitry 110.

The term "processor" as used in the description refers to, for example, circuitry such as a central processing unit (CPU), a graphics processing unit (GPU), an application specific integrated circuit (ASIC), or a programmable logic device (e.g., a simple programmable logic device (SPLD), a complex programmable logic device (CPLD), or a field programmable gate array (FPGA)). The processor reads a computer program stored in the storage circuitry 170 and executes the read computer program to implement the function. Instead of storing a computer program in the storage circuitry 170, the computer program may be embedded directly into the circuitry of the processor. In this case, the processor reads and executes the computer program embedded in the circuitry to implement the function. Each processor in the present embodiment is not limited to a case where each processor is configured as a single circuit. A single processor may be configured by combining a plurality of independent circuits to implement its functions. Furthermore, a plurality of circuitry in FIG. 1 (e.g., B-mode processing circuitry 130, Doppler processing circuitry 140, image generation circuitry 150, and control circuitry 180) may be integrated into a single processor to implement its functions. In other words, the B-mode processing circuitry 130, the Doppler processing circuitry 140, the image generation circuitry 150, and the control circuitry 180 may be integrated into single processing circuitry implemented by a processor. The transmission/reception circuitry 110, the B-mode processing circuitry 130, the Doppler processing circuitry 140, the image generation circuitry 150, and the control circuitry 180 may be integrated into single processing circuitry that includes a processor.

The overall configuration of the ultrasonic diagnostic apparatus 1 according to the first embodiment has been described above.

In the first embodiment, in the ultrasonic diagnostic apparatus 1, a normal mode is set as the initial scanning mode. In the normal mode, the transmission/reception circuitry 110 alternately executes first ultrasonic scanning (first ultrasonic scan) and second ultrasonic scanning (second ultrasonic scan) for the subject P through the ultrasound probe 101. The scanning form of the first ultrasonic scanning is a scanning form in which a first region (first range) formed by a plurality of scanning lines is divided into a plurality of sub-regions (first sub-regions), and ultrasonic transmission/reception is performed for each of the first sub-regions. The first region is, for example, a region inside the subject P. In the present embodiment, the ultrasonic diagnostic apparatus 1 executes the CFM method described above on the data sequence of reflected wave data obtained by first ultrasonic scanning to generate color Doppler image data for each first sub-region.

The transmission/reception circuitry 110 executes first ultrasonic scanning using ultrasonic transmission/reception conditions for collecting color Doppler image data. Then, each time color Doppler image data for each first sub-region is newly generated, the control circuitry 180 displays a color Doppler image based on the newly generated color Doppler image data in a region corresponding to the newly generated color Doppler image data among all regions of the color Doppler image displayed on the display 103. If a color Doppler image is already displayed in the region corresponding to the newly generated color Doppler image data on the display 103, the control circuitry 180 updates the display content in the region corresponding to the newly generated color Doppler image data with a color Doppler image based on the newly generated color Doppler image data.

In a usual color Doppler method, ultrasonic transmission/reception is performed multiple times in the same direction, and blood flow signals are extracted from the thus received signals. A data sequence of reflected wave signals (reflected wave data) from the same location obtained by such ultrasonic transmission/reception is referred to as a packet. The packet size is the number of ultrasonic transmissions / receptions performed in the same direction to obtain blood flow information in one frame. The packet size in a common color Doppler method is variable, for example, from 5 to 16. The performance of the eigenvector MTI filter improves with a larger packet size, but the frame rate decreases as the packet size increases.

The ultrasonic diagnostic apparatus 1 can perform processing in the frame direction (time direction) for the data sequence at the same location in each frame. For example, the ultrasonic diagnostic apparatus 1 can change the MTI filter processing from processing of finite-length data, called packets, to processing of infinite-length data. As a result, the ultrasonic diagnostic apparatus 1 can improve the performance of the MTI filter and thus can detect blood flow information about low-velocity blood flow.

The control circuitry 180 according to the first embodiment executes the first ultrasonic scanning as well as the second ultrasonic scanning for the subject P in a scanning form described below.

The control circuitry 180 divides a second region (second range) formed by a plurality of scanning lines into a plurality of sub-regions (second sub-regions) and allows the ultrasound probe 101 to execute second ultrasonic scanning for each of the second sub-regions through the transmission/reception circuitry 110 in a time division manner during the first ultrasonic scanning. The second region is, for example, a region inside the subject P. The scanning form of the second ultrasonic scanning is a scanning form in which ultrasonic transmission/reception is performed in each of the second sub-regions. The transmission/reception circuitry 110 executes second ultrasonic scanning using ultrasonic transmission/reception conditions for collecting B-mode image data.

Then, each time B-mode image data for each second sub-region is newly generated, the control circuitry 180 displays a B-mode image based on the newly generated B-mode image data in a region corresponding to the newly generated B-mode image data among all regions of the B-mode image displayed on the display 103. If a B-mode image is already displayed in the region corresponding to the newly generated B-mode image data on the display 103, the control circuitry 180 updates the display content in the region corresponding to the newly generated B-mode image data with a B-mode image based on the newly generated B-mode image data.

As described above, the transmission/reception circuitry 110 alternately executes first ultrasonic scanning for each of the first sub-regions and second ultrasonic scanning for each of the second sub-regions through the ultrasound probe 101. With such a scanning form, the ultrasonic diagnostic apparatus 1 according to the first embodiment can set ultrasonic transmission/reception conditions (image quality conditions) independently for the first ultrasonic scanning and the second ultrasonic scanning.

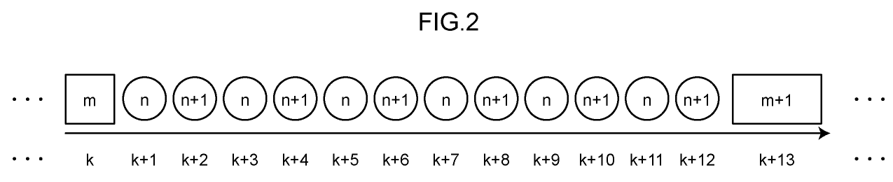

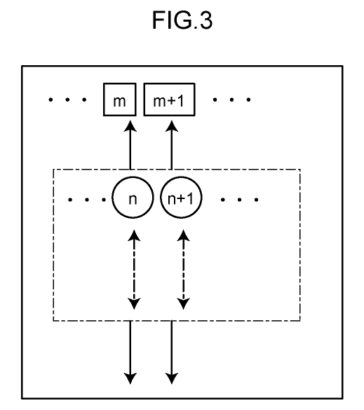

An example of a transmission and reception sequence of ultrasonic waves in the first ultrasonic scanning and the second ultrasonic scanning in the normal mode will be described. FIG. 2 is a diagram for explaining an example of the first ultrasonic scanning and the second ultrasonic scanning in the normal mode according to the first embodiment. FIG. 3 is a diagram illustrating an example of the positions of a plurality of scanning lines (positions in spatial coordinates of image data) in each scan of the first ultrasonic scanning and the second ultrasonic scanning according to the first embodiment.

In FIG. 2, k, k+1, k+2, ..., k+12, k+13 indicate the order of transmission and reception of ultrasonic waves. k is an integer equal to or greater than 1. In FIG. 2, 12 circular frames arranged in a row indicate the first ultrasonic scanning for the first sub-region. In FIG. 2, two rectangular frames indicate the second ultrasonic scanning for the second sub-region. Furthermore, n or n+1 in the circular frame is an identifier that indicates a scanning line in the first sub-region to which ultrasonic waves are transmitted and received by the first ultrasonic scanning, and is a value that indicates the order of the scanning line in the first sub-region and includes information indicating the position of the scanning line in spatial coordinates. Furthermore, m or m+1 in the rectangular frame is an identifier that indicates a scanning line in the second sub-region to which ultrasonic waves are transmitted and received by the second ultrasonic scanning, and is a value that indicates the order of the scanning line in the second sub-region and includes information indicating the position of the scanning line in spatial coordinates. Here, n is an odd number equal to or greater than 1 and equal to or less than N, as described below. Furthermore, m is an integer equal to or greater than 1 and less than M, as described below. In the following description, the scanning line in the first sub-region identified by an identifier s (s is an integer) is denoted as "first scanning line s". Similarly, the scanning line in the second sub-region identified by an identifier t (t is an integer) is denoted as "second scanning line t".

In the example illustrated in FIGS. 2 and 3, the first region is formed by N scanning lines (rasters), the second region is formed by M scanning lines, the first region is divided into (N/2) first sub-regions, and the second region is divided into M second sub-regions. In other words, the first region is the region including N scanning lines including a first scanning line 1, a first scanning line 2, ..., a first scanning line N, and the second region is the region including M scanning lines including a second scanning line 1, a second scanning line 2, ..., a second scanning line M. Here, N is an integer equal to or greater than 2 and a multiple of 2. M is an integer greater than 1. In addition, "/" is an operator indicating division. Thus, in the example illustrated in FIGS. 2 and 3, each of a plurality of first sub-regions is formed by two first scanning lines, and each of a plurality of second sub-regions is formed by one scanning line.

As illustrated in FIG. 2, the transmission/reception circuitry 110 scans the second sub-region including the second scanning line m by transmitting and receiving ultrasonic waves along the second scanning line m as the kth transmission/reception. B-mode image data corresponding to the second sub-region including the second scanning line m is then generated from the reflected wave data obtained by scanning the second sub-region including the second scanning line m. As used herein, transmitting and receiving ultrasonic waves along a certain scanning line is synonymous with executing transmission and reception of ultrasonic waves for a certain scanning line.

The transmission/reception circuitry 110 transmits and receives ultrasonic waves along the first scanning line n as the (k+1)th transmission/reception, and transmits and receives ultrasonic waves along the first scanning line n+1 as the (k+2)th transmission/reception. As illustrated in FIG. 2, the transmission/reception circuitry 110 then repeats transmitting and receiving ultrasonic waves along the first scanning line n and transmitting and receiving ultrasonic waves along the first scanning line n+1, from the (k+3)th transmission/reception to the (k+12)th transmission/reception. In this way, since ultrasonic waves are transmitted and received along the two first scanning lines n and n+1 alternately, the number of alternating stages indicating the number of scanning lines along which ultrasonic waves are transmitted and received alternately is two. The group of two first scanning lines n and n+1 along which ultrasonic waves are transmitted and received alternately is called an alternating stage group. In this way, the transmission/reception circuitry 110 scans the first sub-region including the first scanning line n and the first scanning line n+1. This results in six pieces of reflected wave data corresponding to the first scanning line n and six pieces of reflected wave data corresponding to the first scanning line n+1.

A data sequence including six pieces of reflected wave data corresponding to the first scanning line n is then input to the eigenvector MTI filter to obtain color Doppler data. Similarly, a data sequence including six pieces of reflected wave data corresponding to the first scanning line n+1 is input to the eigenvector MTI filter to obtain color Doppler data. In other words, the packet size of the eigenvector MTI filter in this case is six. Color Doppler image data corresponding to the first sub-region including the first scanning line n and the first scanning line n+1 is then generated from these pieces of obtained color Doppler data.

The transmission/reception circuitry 110 then scans the second sub-region including the second scanning line m+1 by transmitting and receiving ultrasonic waves along the second scanning line m+1 as the (k+13)th transmission/reception. B-mode image data corresponding to the second sub-region including the second scanning line m+1 is then generated from the reflected wave data obtained by scanning the second sub-region including the second scanning line m+1.

The transmission/reception circuitry 110 then alternately executes the first ultrasonic scanning and the second ultrasonic scanning so that all first sub-regions and all second sub-regions are scanned, that is, the entire first region and the entire second region are scanned. This results in color Doppler image data for one frame corresponding to the first region and B-mode image data for one frame corresponding to the second region. The transmission/reception circuitry 110 then repeats scanning the entire first region and the entire second region multiple times. As a result, color Doppler image data for a plurality of frames and B-mode image data for a plurality of frames are obtained, and on the display 103, a B-mode image is displayed as a moving image in real time and a color Doppler image superimposed on the B-mode image is displayed as a moving image in real time.

Here, in the normal mode, the first ultrasonic scanning and the second ultrasonic scanning are executed alternately, but a residual signal (residual echo) of the ultrasonic wave transmitted last in the second ultrasonic scanning may enter a reception period of the reflected wave of the ultrasonic wave transmitted first in the first ultrasonic scanning. The reason for this may be that the transmission/reception circuitry 110 (ultrasound probe 101) transmits an ultrasonic wave in the first ultrasonic scanning before receiving the reflected wave from the depth of the ultrasonic wave transmitted last in the second ultrasonic scanning.

For example, in the example illustrated in FIG. 2, the residual signal of the ultrasonic wave transmitted along the second scanning line m as the kth transmission/reception enters the reception period of the reflected wave of the ultrasonic wave transmitted along the first scanning line n as the (k+1)th transmission/reception. Therefore, the reflected wave signal on the first scanning line n obtained by the (k+1)th transmission/reception output from the ultrasound probe 101 contains the residual signal and therefore is affected by the residual signal.

Here, the residual signals of ultrasonic waves transmitted along the first scanning line n+1 as the (k+2)th transmission/reception, (k+4)th transmission/reception, (k+6)th transmission/reception, ..., and (k+10)th transmission/reception enter the reception periods of the reflected waves of the ultrasonic waves transmitted along the first scanning line n as the (k+3)th transmission/reception, (k+5)th transmission/reception, (k+7)th transmission/reception, ..., and (k+11)th transmission/reception, respectively. However, the residual signals contained in the reflected wave signal on the first scanning line n obtained by the (k+3)th transmission/reception, the reflected wave signal on the first scanning line n obtained by the (k+5)th transmission/reception, the reflected wave signal on the first scanning line n obtained by the (k+7)th transmission/reception, ..., and the reflected wave signal on the first scanning line n obtained by the (k+11)th transmission/reception, output from the ultrasound probe 101, are similar signals because the transmission/reception that is a source of the residual signals is based on the same ultrasonic transmission/reception conditions (ultrasonic transmission/reception conditions for collecting color Doppler image data). Therefore, the residual signals contained in the reflected wave signals on the first scanning line n obtained by the (k+3)th transmission/reception, (k+5)th transmission/reception, (k+7)th transmission/reception, ..., and (k+11)th transmission/reception are unlikely to cause artifacts in a color Doppler image.

On the other hand, the residual signal contained in the reflected wave signal on the first scanning line n obtained by the (k+1)th transmission/reception output from the ultrasound probe 101 is a signal generated not by the transmission/reception based on the ultrasonic transmission/reception conditions for collecting color Doppler image data but by the second ultrasonic scan based on the ultrasonic transmission/reception conditions for collecting B-mode image data. Therefore, the aspect of the residual signal contained in the reflected wave signal on the first scanning line n obtained by the (k+1)th transmission/reception is significantly different from the aspect of the residual signals contained in the reflected wave signals on the first scanning line n obtained by the (k+3)th transmission/reception, (k+5)th transmission/reception, (k+7)th transmission/reception, ..., and (k+11)th transmission/reception. Therefore, the residual signal contained in the reflected wave signal on the first scanning line n obtained by the (k+1)th transmission/reception is imaged as an artifact image and becomes a cause of artifacts in the color Doppler image.

Note that the residual signals of ultrasonic waves transmitted along the first scanning line n as the (k+1)th transmission/reception, (k+3)th transmission/reception, (k+5)th transmission/reception, ..., and (k+11)th transmission/reception enter the reception periods of the reflected waves of the ultrasonic waves transmitted along the first scanning line n+1 as the (k+2)th transmission/reception, (k+4)th transmission/reception, (k+6)th transmission/reception, ..., and (k+12)th transmission/reception, respectively. However, the residual signals contained in the reflected wave signal on the first scanning line n+1 obtained by the (k+2)th transmission/reception, the reflected wave signal on the first scanning line n+1 obtained by the (k+4)th transmission/reception, the reflected wave signal on the first scanning line n+1 obtained by the (k+6)th transmission/reception, ..., and the reflected wave signal on the first scanning line n+1 by the (k+12)th transmission/reception, output from the ultrasound probe 101, are similar signals because the transmission/reception that is a source of the residual signals is based on the same ultrasonic transmission/reception conditions (ultrasonic transmission/reception conditions for collecting color Doppler image data). Therefore, the residual signals contained in the reflected wave signals on the first scanning line n+1 obtained by the (k+2)th transmission/reception, (k+4)th transmission/reception, (k+6)th transmission/reception, ..., and (k+12)th transmission/reception are unlikely to cause artifacts in a color Doppler image.

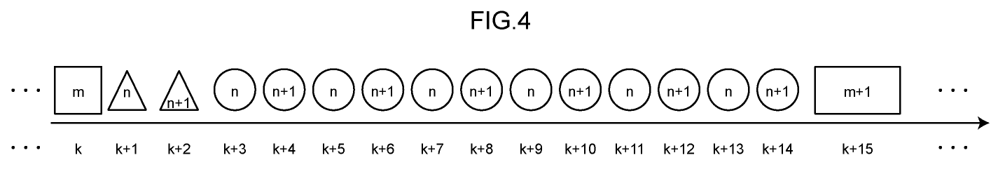

As described above, the residual signal contained in the reflected wave signal on the first scanning line n obtained by the (k+1)th transmission/reception is imaged as an artifact image and becomes a cause of artifacts in the color Doppler image. Therefore, it is conceivable to perform dummy rate transmission to reduce residual multiplex. The ultrasonic diagnostic apparatus that performs such dummy rate transmission will be described as the ultrasonic diagnostic apparatus according to a comparative example. FIG. 4 is a diagram for explaining dummy rate transmission executed by the ultrasonic diagnostic apparatus according to a comparative example when a dummy rate mode is set as a scanning mode. In the description of the transmission/reception sequence of ultrasonic waves in dummy rate transmission in the dummy rate mode illustrated in FIG. 4, the points that differ from the transmission/reception sequence of ultrasonic waves in the normal mode illustrated in FIG. 2 will mainly be explained.

In FIG. 4, k, k+1, k+2, ..., k+14, k+15 indicate the order of transmission and reception of ultrasonic waves. In FIG. 4, two triangular frames and twelve circular frames aligned in a row indicate the first ultrasonic scanning for the first sub-region. Furthermore, n or n+1 in the triangular frame is an identifier that indicates a scanning line in the first sub-region to which ultrasonic waves are transmitted by the first ultrasonic scanning, and is a value that indicates the order of the scanning line in the first sub-region and includes information indicating the position of the scanning line in spatial coordinates.

As illustrated in FIG. 4, the ultrasonic diagnostic apparatus according to the comparative example scans the second sub-region including the second scanning line m by transmitting and receiving ultrasonic waves along the second scanning line m as the kth transmission/reception. The ultrasonic diagnostic apparatus then generates B-mode image data corresponding to the second sub-region including the second scanning line m from the reflected wave data obtained by scanning the second sub-region including the second scanning line m.

The ultrasonic diagnostic apparatus then transmits an ultrasonic wave along the first scanning line n as the (k+1)th transmission/reception and transmits an ultrasonic wave along the first scanning line n+1 as the (k+2)th transmission/reception. However, the reception circuitry of the ultrasonic diagnostic apparatus does not generate reflected wave data from the reflected wave of the ultrasonic wave obtained in each of the (k+1)th transmission/reception and the (k+2)th transmission/reception. Alternatively, the circuitry on the subsequent stage to the reception circuitry of the ultrasonic diagnostic apparatus does not generate data using the reflected wave data generated from the reflected wave of the ultrasonic wave obtained in each of the (k+1)th transmission/reception and the (k+2)th transmission/reception.

As illustrated in FIG. 4, the ultrasonic diagnostic apparatus then repeats transmitting and receiving ultrasonic waves along the first scanning line n and transmitting and receiving ultrasonic waves along the first scanning line n+1, from the (k+3)th transmission/reception to the (k+14)th transmission/reception. In this way, the ultrasonic diagnostic apparatus scans the first sub-region including the first scanning line n and the first scanning line n+1. This results in six pieces of reflected wave data corresponding to the first scanning line n and six pieces of reflected wave data corresponding to the first scanning line n+1.

The ultrasonic diagnostic apparatus then inputs a data sequence including six pieces of reflected wave data corresponding to the first scanning line n to the eigenvector MTI filter to obtain color Doppler data. Similarly, the ultrasonic diagnostic apparatus inputs a data sequence including six pieces of reflected wave data corresponding to the first scanning line n+1 to the eigenvector MTI filter to obtain color Doppler data. In other words, the packet size of the eigenvector MTI filter in this case is six. The ultrasonic diagnostic apparatus then generates color Doppler image data corresponding to the first sub-region including the first scanning line n and the first scanning line n+1 from these pieces of obtained color Doppler data.

The ultrasonic diagnostic apparatus then scans the second sub-region including the second scanning line m+1 by transmitting and receiving ultrasonic waves along the second scanning line m+1 as the (k+15)th transmission/reception. The ultrasonic diagnostic apparatus then generates B-mode image data corresponding to the second sub-region including the second scanning line m+1 from the reflected wave data obtained by scanning the second sub-region including the second scanning line m+1.

The ultrasonic diagnostic apparatus then alternately executes the first ultrasonic scanning and the second ultrasonic scanning so that all first sub-regions and all second sub-regions are scanned, that is, the entire first region and the entire second region are scanned. This results in color Doppler image data for one frame corresponding to the first region and B-mode image data for one frame corresponding to the second region. The ultrasonic diagnostic apparatus then repeats scanning the entire first region and the entire second region multiple times. As a result, color Doppler image data for a plurality of frames and B-mode image data for a plurality of frames are obtained, and on the display of the ultrasonic diagnostic apparatus, a B-mode image is displayed as a moving image in real time and a color Doppler image superimposed on the B-mode image is displayed as a moving image in real time.

Here, the residual signal of the ultrasonic wave transmitted along the second scanning line m as the kth transmission/reception enters the reception period of the reflected wave of the ultrasonic wave transmitted along the first scanning line n as the (k+1)th transmission/reception. However, in the ultrasonic diagnostic apparatus according to the comparative example, the reflected wave signal obtained by the (k+1)th transmission/reception does not contribute to the generation of color Doppler image data.

To illustrate with a specific example, in the example in FIG. 4, the residual signals of ultrasonic waves transmitted along the first scanning line n+1 as the (k+2)th transmission/reception, (k+4)th transmission/reception, (k+6)th transmission/reception, ..., and (k+12)th transmission/reception enter the reception periods of the reflected waves of the ultrasonic waves transmitted along the first scanning line n as the (k+3)th transmission/reception, (k+5)th transmission/reception, (k+7)th transmission/reception, ..., and (k+13)th transmission/reception, respectively. However, the residual signals contained in the reflected wave signal on the first scanning line n obtained by the (k+3)th transmission/reception, the reflected wave signal on the first scanning line n obtained by the (k+5)th transmission/reception, the reflected wave signal on the first scanning line n obtained by the (k+7)th transmission/reception, ..., and the reflected wave signal on the first scanning line n obtained by the (k+13)th transmission/reception, output from the ultrasound probe 101, are similar signals because the transmission/reception that is a source of the residual signals is based on the same ultrasonic transmission/reception conditions (ultrasonic transmission/reception conditions for collecting color Doppler image data). Therefore, the residual signals contained in the reflected wave signals on the first scanning line n obtained by the (k+3)th transmission/reception, (k+5)th transmission/reception, (k+7)th transmission/reception, ..., and (k+13)th transmission/reception are unlikely to cause artifacts in a color Doppler image.

In the example in FIG. 4, the ultrasonic diagnostic apparatus according to the comparative example may omit the (k+1)th transmission/reception and not perform the (k+1)th transmission/reception. However, because of the difficulties in sequence and data handling, and the difficulty in assuming the degree of residual multiplexing in advance, it is preferable that the ultrasonic diagnostic apparatus according to the comparative example performs the (k+1)th transmission/reception.

The ultrasonic diagnostic apparatus according to the comparative example can reduce the effect of residual multiplex on color Doppler image data. However, as illustrated in FIG. 4, when color Doppler image data corresponding to one first sub-region is generated, 14 transmissions / receptions are required from the (k+1)th transmission/reception to the (k+14)th transmission/reception. Therefore, the frame rate of color Doppler image data is reduced in the ultrasonic diagnostic apparatus according to the comparative example.

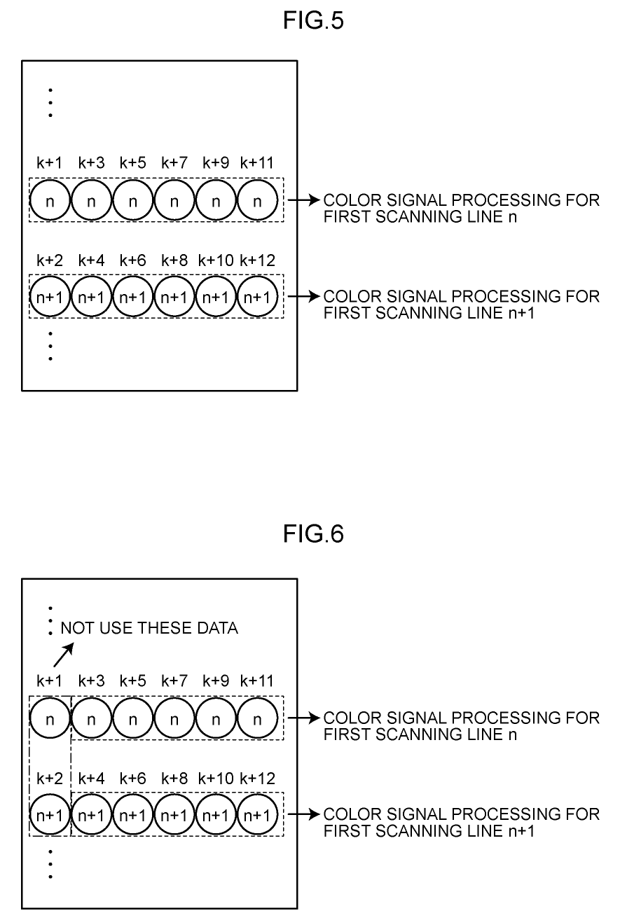

FIG. 5 is a diagram illustrating an example of a data sequence input to the eigenvector MTI filter in the normal mode illustrated in FIG. 2. As illustrated in FIG. 5, in the normal mode, six pieces of reflected wave data obtained by the (k+1)th transmission/reception, (k+3)th transmission/reception, ..., (k+9)th transmission/reception, and (k+11)th transmission/reception, and six pieces of reflected wave data obtained by the (k+2)th transmission/reception, (k+4)th transmission/reception, ..., (k+10)th transmission/reception, and (k+12)th transmission/reception are input to the eigenvector MTI filter. This results in color Doppler image data corresponding to the first sub-region including the first scanning line n and the first scanning line n+1.

Here, the packet size of the eigenvector MTI filter is variable. Using the variable packet size, the ultrasonic diagnostic apparatus 1 according to the first embodiment then executes the processing described below to reduce the effect of residual multiplex on the color Doppler image data while suppressing the reduction in the frame rate of the color Doppler image data.

FIG. 6 is a diagram illustrating an example of a data sequence input to the eigenvector MTI filter in a residual multiplex reduction mode according to the first embodiment. The ultrasonic diagnostic apparatus 1 is set to the residual multiplex reduction mode when the residual multiplex reduction button described above is pressed by the operator. In this way, the ultrasonic diagnostic apparatus 1 transitions to the residual multiplex reduction mode when the residual multiplex reduction button is pressed. In the residual multiplex reduction mode, the transmission/reception circuitry 110 alternately executes the first ultrasonic scanning and the second ultrasonic scanning in the same way that the first ultrasonic scanning and the second ultrasonic scanning are executed alternately in the normal mode.

However, as illustrated in FIG. 6, in the residual multiplex reduction mode, among six pieces of reflected wave data obtained by the (k+1)th transmission/reception, (k+3)th transmission/reception, ..., (k+9)th transmission/reception, and (k+11)th transmission/reception, five pieces of reflected wave data obtained by the (k+3)th transmission/reception, ..., (k+9)th transmission/reception, and (k+11)th transmission/reception are input to the eigenvector MTI filter. Further, among six pieces of reflected wave data obtained by the (k+2)th transmission/reception, (k+4)th transmission/reception, ..., (k+10)th transmission/reception, and (k+12)th transmission/reception, five pieces of reflected wave data obtained by the (k+4)th transmission/reception, ..., (k+10)th transmission/reception, and (k+12)th transmission/reception are input to the eigenvector MTI filter. In other words, the packet size of the eigenvector MTI filter in the residual multiplex reduction mode is five. This results in color Doppler image data corresponding to the first sub-region including the first scanning line n and the first scanning line n+1.

In this way, in the residual multiplex reduction mode, the ultrasonic diagnostic apparatus 1 generates color Doppler image data without using reflected wave data affected by residual multiplex that becomes a cause of artifacts, such as reflected wave data based on the reflected wave signal on the first scanning line n obtained by the (k+1)th transmission / reception. Therefore, in the residual multiplex reduction mode, the ultrasonic diagnostic apparatus 1 can reduce the effect of residual multiplex on color Doppler image data.

In the residual multiplex reduction mode, the ultrasonic diagnostic apparatus 1 can generate color Doppler image data corresponding to one first sub-region by 12 transmissions / receptions from the (k+1)th transmission / reception to the (k+12)th transmission/reception by setting the packet size of the eigenvector MTI filter to five. Therefore, the ultrasonic diagnostic apparatus 1 can suppress the reduction in the frame rate of color Doppler image data.

Here, in the ultrasonic diagnostic apparatus 1, "ten times (10)" is set as a predetermined reference value as the number of transmissions / receptions to obtain the reflected wave signals used in generating color Doppler image data corresponding to one first sub-region. The predetermined reference value is less than the number of transmissions / receptions "12 times" to obtain the reflected wave signals used in generating color Doppler image data corresponding to one first sub-region in the dummy rate mode described above. In the first ultrasonic scanning in the residual multiplex reduction mode, the ultrasonic diagnostic apparatus 1 executes the first ultrasonic scanning so that the reflected wave signals used in generating color Doppler image data corresponding to one first sub-region are obtained with a number of transmissions / receptions that is equal to or less than the predetermined reference value "10 times". For example, the ultrasonic diagnostic apparatus 1 generates, as at least part of color Doppler image data representing the entire first region, color Doppler image data representing a region (one first sub-region) in the first region that corresponds to 10 transmissions / receptions, based on 10 reflected wave signals obtained by 10 transmissions / receptions (10 transmissions / receptions from the (k+3)th transmission / reception to the (k+12)th transmission / reception), which is equal to or less than the predetermined reference value "10 times". Here, the 10 transmissions / receptions from the (k+3)th transmission / reception to the (k+12)th transmission/reception is, for example, an example of a second predetermined number of transmissions / receptions.

Therefore, the ultrasonic diagnostic apparatus 1 according to the first embodiment can reduce the effect of residual multiplex on color Doppler image data while suppressing the reduction in the frame rate of color Doppler image data.

In the residual multiplex reduction mode, the ultrasonic diagnostic apparatus 1 may omit the (k+2)th transmission/reception and not perform the (k+2)th transmission/reception. However, because of the difficulties in data handling and the like, it is preferable that the ultrasonic diagnostic apparatus 1 performs the (k+1)th transmission/reception.

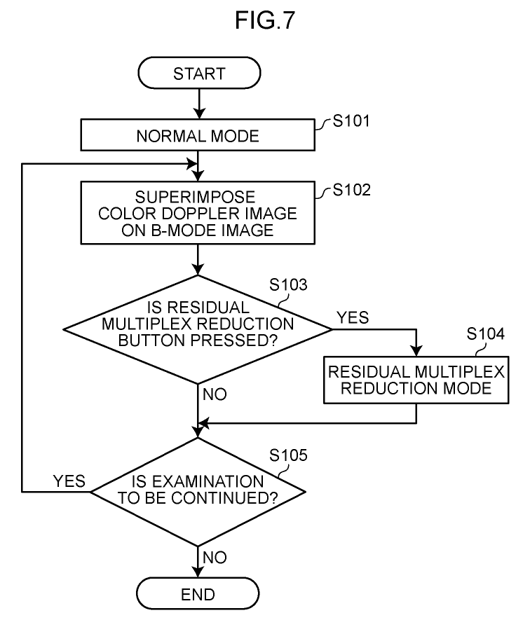

The flow of exemplary processing executed by the ultrasonic diagnostic apparatus 1 according to the first embodiment will now be described. FIG. 7 is a flowchart illustrating the flow of exemplary processing executed by the ultrasonic diagnostic apparatus 1 according to the first embodiment. The processing illustrated in FIG. 7 is executed when the operator operates the input device 102 and inputs an instruction to the control circuitry 180 to superimpose a color Doppler image on a B-mode image for the examination of the subject P.

As illustrated in FIG. 7, the ultrasonic diagnostic apparatus 1 alternately executes first ultrasonic scanning and second ultrasonic scanning in the normal mode (see FIG. 2) for the subject P to generate B-mode image data and color Doppler image data (step S101).

The ultrasonic diagnostic apparatus 1 then superimposes a color Doppler image based on the color Doppler image data on a B-mode image based on the B-mode image data generated in the set scanning mode, and displays the images on the display 103 (step S102). The scanning mode can be the normal mode initially set or the residual multiplex reduction mode set at step S104.

The control circuitry 180 of the ultrasonic diagnostic apparatus 1 then determines whether the residual multiplex reduction button has been pressed by the operator (step S103). If it is determined that the residual multiplex reduction button has been pressed by the operator (Yes at step S103), the control circuitry 180 proceeds to step S104. As used herein, pressing the residual multiplex reduction button is synonymous with turning on the residual multiplex reduction button. On the other hand, if it is determined that the residual multiplex reduction button is not pressed by the operator (No at step S103), the control circuitry 180 proceeds to step S105.

The ultrasonic diagnostic apparatus 1 alternately executes first ultrasonic scanning and second ultrasonic scanning in the residual multiplex reduction mode for the subject P to generate B-mode image data and color Doppler image data (step S104), and then proceeds to step S105.

The control circuitry 180 of the ultrasonic diagnostic apparatus 1 then determines whether to continue the examination (step S105). For example, at step S105, the control circuitry 180 determines whether an instruction to terminate the examination of the subject P (termination instruction) has been input to the control circuitry 180 by the operator operating the input device 102. If it is determined that no termination instruction is input, the control circuitry 180 determines to continue the examination (Yes at step S105), and returns to step S102.

On the other hand, if it is determined that a termination instruction has been input, the control circuitry 180 determines not to continue the examination (No at step S105), and terminates the processing illustrated in FIG. 7.

In the processing illustrated in FIG. 7, when the residual multiplex reduction button that has been turned on is turned off by the operator, that is, when the residual multiplex reduction button is released, the ultrasonic diagnostic apparatus 1 alternately executes first ultrasonic scanning and second ultrasonic scanning in the normal mode for the subject P to generate B-mode image data and color Doppler image data.

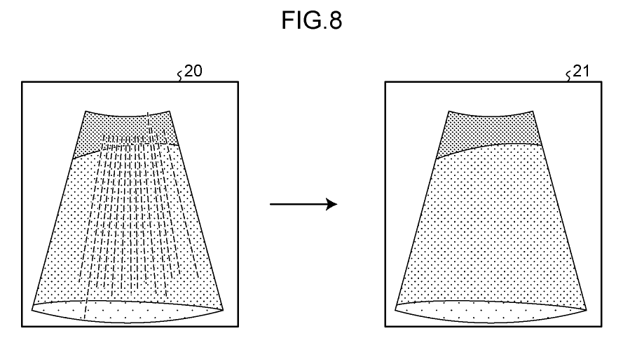

FIG. 8 is a diagram illustrating an example of a color Doppler image displayed by the ultrasonic diagnostic apparatus 1 according to the first embodiment. FIG. 8 illustrates a color Doppler image 20 displayed on the display 103 in the normal mode and a color Doppler image 21 displayed on the display 103 in the residual multiplex reduction mode.

Comparing the color Doppler image 20 with the color Doppler image 21, it can be understood that artifacts due to the effect of residual multiplex occur in the color Doppler image 20, while the occurrence of artifacts is suppressed in the color Doppler image 21.

The ultrasonic diagnostic apparatus 1 according to the first embodiment has been described above.

As described above, the ultrasonic diagnostic apparatus 1 according to the first embodiment includes a scanning unit that repeats alternately executing two ultrasonic scannings: first ultrasonic scanning that repeats transmission and reception on at least one first scanning line included in a first region under a first ultrasonic transmission condition; and second ultrasonic scanning that performs transmission and reception on at least one second scanning line included in a second region under a second ultrasonic transmission condition. As a result, the scanning unit performs transmission and reception on all first scanning lines in the first region and all second scanning lines in the second region. The scanning unit includes, for example, the ultrasound probe 101, the transmission/reception circuitry 110, and the control circuitry 180, and is implemented by the ultrasound probe 101, the transmission / reception circuitry 110, and the control circuitry 180. However, the scanning unit may further include circuitry and/or equipment other than these.

The ultrasonic diagnostic apparatus 1 also includes a generation unit that generates Doppler image data representing the first region, based on reflected wave signals obtained by transmission and reception on all the first scanning lines in the first region, and generates B-mode image data representing the second region, based on reflected wave signals obtained by transmission and reception on all the second scanning lines in the second region. The generation unit includes, for example, the transmission / reception circuitry 110, the B-mode processing circuitry 130, the Doppler processing circuitry 140, the image generation circuitry 150, and the control circuitry 180, and is implemented by the transmission/reception circuitry 110, the B-mode processing circuitry 130, the Doppler processing circuitry 140, the image generation circuitry 150, and the control circuitry 180. However, the generation unit may further include circuitry and/or equipment other than these. Here, the reflected wave signal is, for example, an example of a signal. The Doppler image data is, for example, an example of first image data. The B-mode image data is, for example, an example of second image data.

When the first ultrasonic scanning is executed by the scanning unit after the second ultrasonic scanning in the residual multiplex reduction mode, the generation unit generates, as at least part of color Doppler image data representing the entire first region, color Doppler image data representing a region (one first sub-region) in the first region that corresponds to 10 transmissions / receptions, based on 10 reflected wave signals obtained by 10 transmissions / receptions (10 transmissions / receptions from the (k+3)th transmission / reception to the (k+12)th transmission/reception), which is equal to or less than a predetermined reference value "10 times", excluding a first predetermined number of transmissions / receptions from the first transmission / reception (in the example in FIG. 6, two transmissions / receptions from the first transmission / reception (the (k+1)th transmission / reception and the (k+2)th transmission / reception)), among all transmissions / receptions in the first ultrasonic scanning. The residual multiplex reduction mode is, for example, an example of a first scanning mode.

When the first ultrasonic scanning is executed by the scanning unit after the second ultrasonic scanning in the residual multiplex reduction mode, the generation unit generates, as at least part of color Doppler image data representing the first region, color Doppler image data representing the first sub-region corresponding to the 10 transmissions / receptions, using the 10 reflected wave signals obtained by 10 transmissions / receptions (10 transmissions / receptions from the (k+3)th transmission/reception to the (k+12)th transmission / reception), which is equal to or less than the predetermined reference value "10 times", and the eigenvector MTI filter.

As described above, the ultrasonic diagnostic apparatus 1 according to the first embodiment includes a collection unit that collects a plurality of pieces of reflected wave data by performing ultrasonic transmission/reception multiple times on the same scanning line. The collection unit includes, for example, the ultrasound probe 101, the transmission/reception circuitry 110, and the control circuitry 180, and is implemented by the ultrasound probe 101, the transmission / reception circuitry 110, and the control circuitry 180. However, the collection unit may further include circuitry and/or equipment other than these. The generation unit generates a color Doppler image using a plurality of pieces of reflected wave data collected by performing ultrasonic transmission/reception multiple times on the same scanning line. The collection unit collects a plurality of pieces of reflected wave data by multiple ultrasonic transmissions / receptions on a predetermined scanning line. In the processing at S104, the generation unit determines the number of pieces of reflected wave data not to be used for color Doppler image generation according to the effect of a reflected wave signal (echo signal) transmitted / received at a time phase prior to the multiple ultrasonic transmissions / receptions on the predetermined scanning line. The generation unit then generates a color Doppler image using a plurality of pieces of reflected wave data, excluding a piece or pieces of reflected wave data corresponding to the determined number of pieces of reflected wave data counted from a first piece of reflected wave data, among the pieces of reflected wave data obtained by the multiple ultrasonic transmissions / receptions on the predetermined scanning line (the pieces of reflected wave data collected by the collection unit). The reflected wave data is, for example, an example of transmission/reception data.

In the first embodiment, in the residual multiplex reduction mode, the generation unit generates a color Doppler image, using a piece or pieces of reflected wave data corresponding to a second number of pieces of reflected wave data which is equal to or less than the predetermined reference number, excluding a piece or pieces of reflected wave data corresponding to a first number of pieces of reflected wave data as the number of pieces of reflected wave data not to be used for color Doppler image generation, counted from the first piece of reflected wave data, among the pieces of reflected wave data collected by multiple ultrasonic transmissions / receptions on the predetermined scanning line.

In the first embodiment, in the residual multiplex reduction mode, the generation unit generates a color Doppler image using a piece or pieces of reflected wave data corresponding to the second number of pieces of reflected wave data and the eigenvector MTI filter.

Therefore, as described above, the ultrasonic diagnostic apparatus 1 can reduce the effect of residual multiplex on color Doppler image data while suppressing the reduction in the frame rate of color Doppler image data.

### Second Embodiment

An ultrasonic diagnostic apparatus according to a second embodiment will now be described. In the description of the second embodiment, the same configuration as in the first embodiment is denoted by the same reference sign and the description thereof may be omitted. In the description of the second embodiment, the points different from the first embodiment will be mainly described.

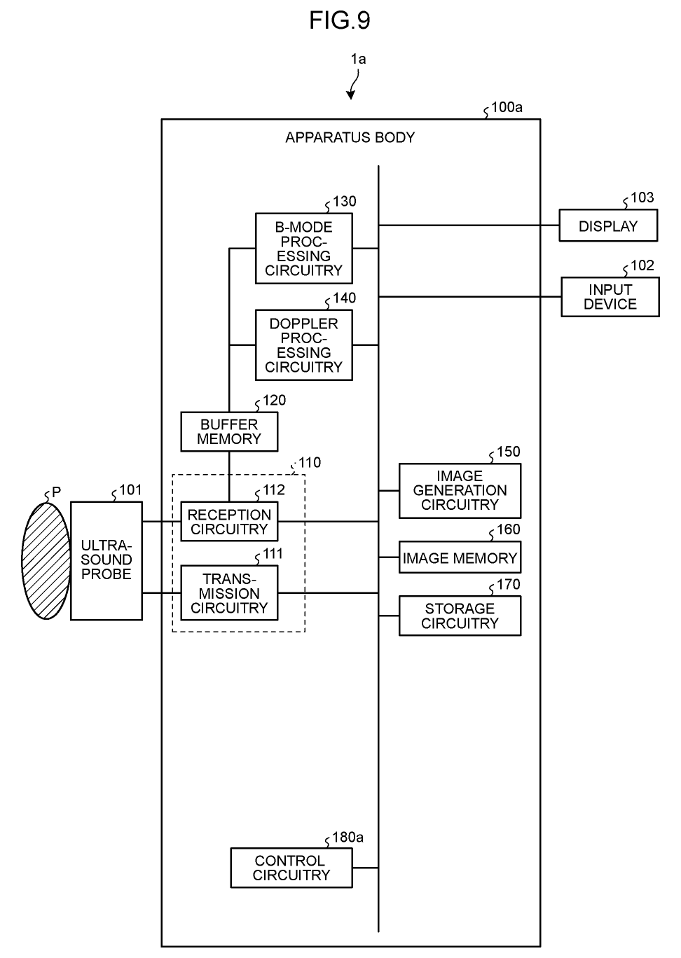

FIG. 9 is a block diagram illustrating an example configuration of an ultrasonic diagnostic apparatus 1a according to the second embodiment. The ultrasonic diagnostic apparatus 1a according to the second embodiment differs from the ultrasonic diagnostic apparatus 1 according to the first embodiment in that it includes an apparatus body 100a instead of the apparatus body 100. The apparatus body 100a according to the second embodiment differs from the apparatus body 100 in that it includes control circuitry 180a instead of the control circuitry 180.

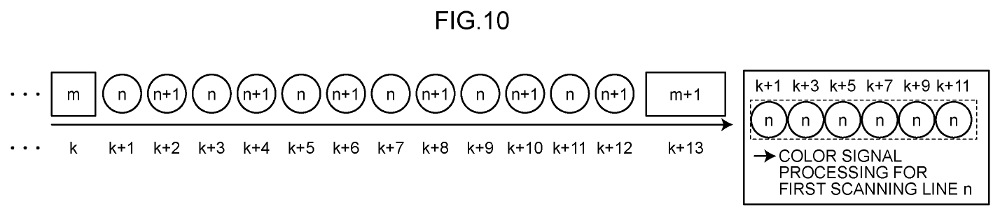

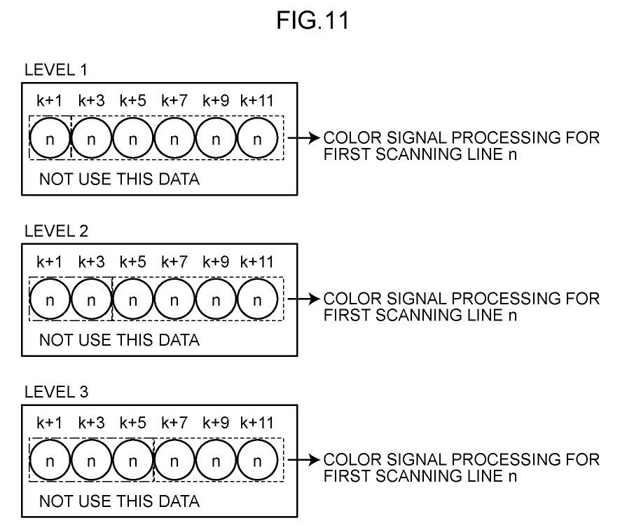

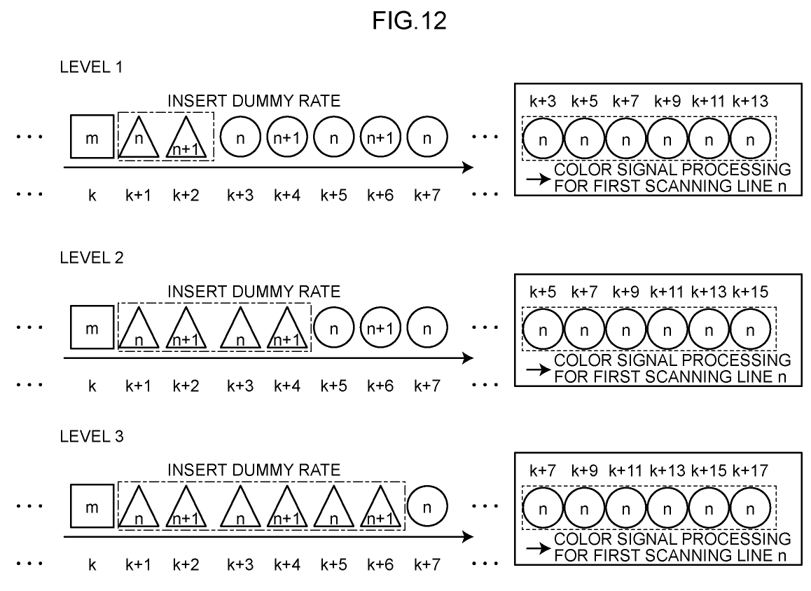

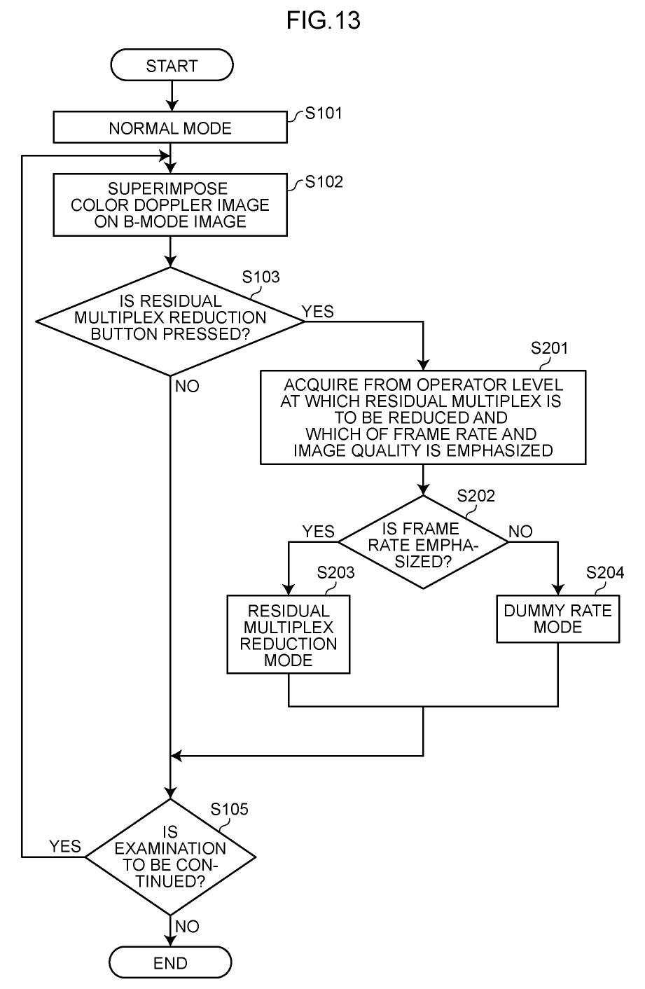

FIG. 10 is a diagram for explaining an example of first ultrasonic scanning and second ultrasonic scanning in the normal mode according to the second embodiment. FIG. 11 is a diagram illustrating an example of a data sequence input to the eigenvector MTI filter in the residual multiplex reduction mode according to the second embodiment. FIG. 12 is a diagram for explaining an example of first ultrasonic scanning and second ultrasonic scanning in the dummy rate mode according to the second embodiment. FIG. 13 is a flowchart illustrating the flow of exemplary processing executed by the ultrasonic diagnostic apparatus 1a according to the second embodiment.

The processing illustrated in FIG. 13 is executed when the operator operates the input device 102 and inputs an instruction to the control circuitry 180a to superimpose a color Doppler image on a B-mode image for the examination of the subject P.

The processing at each of steps S101, S102, S103, and S105 illustrated in FIG. 13 is similar to the processing at each of steps S101, S102, S103, and S105 illustrated in FIG. 7. However, the processing executed by the control circuitry 180 in FIG. 7 is executed by the control circuitry 180a in FIG. 13. For example, at step S101 illustrated in FIG. 13, as illustrated in FIG. 10, the ultrasonic diagnostic apparatus 1a alternately executes first ultrasonic scanning and second ultrasonic scanning in the normal mode for the subject P to generate B-mode image data and color Doppler image data, in the same way as in the ultrasonic diagnostic apparatus 1 according to the first embodiment.

If it is determined that the residual multiplex reduction button has been pressed by the operator (Yes at step S103), the control circuitry 180a proceeds to step S201. On the other hand, if it is determined that the residual multiplex reduction button is not pressed by the operator (No at step S103), the control circuitry 180a proceeds to step S105.

The control circuitry 180a acquires from the operator the degree (level) of residual multiplex reduction and which of the frame rate and the image quality of color Doppler image data is to be emphasized (step S201). For example, at step S201, the control circuitry 180a displays a plurality of levels 1 to 3 on the display 103 with any one level selectable by the operator from the levels 1 to 3 at which residual multiplex is to be reduced. The higher the value of the level, the greater the degree of residual multiplex reduction. At step S201, the control circuitry 180a displays a screen on the display 103 to allow the operator to select which of the frame rate and the image quality of color Doppler image data is to be emphasized. The operator operates the input device 102 to select one of the levels 1 to 3. The operator also operates the input device 102 to select which of the frame rate and the image quality of color Doppler image data is to be emphasized. The control circuitry 180a acquires the selection made by the operator.

The control circuitry 180a then determines whether the operator has selected to emphasize the frame rate of color Doppler image data at step S201 (step S202). Here, the frame rate of color Doppler image data obtained in the residual multiplex reduction mode is higher than the frame rate of color Doppler image data obtained in the dummy rate mode. Therefore, if the operator has selected to emphasize the frame rate of color Doppler image data (Yes at step S202), the ultrasonic diagnostic apparatus 1a sets the scanning mode to the residual multiplex reduction mode, alternately executes first ultrasonic scanning and second ultrasonic scanning in the residual multiplex reduction mode for the subject P to generate B-mode image data and color Doppler image data (step S203), and then proceeds to step S105.

Here, at step S203, the processing of reducing residual multiplex at the level selected by the operator is performed. For example, as illustrated in FIG. 11, at level 1, among six pieces of reflected wave data obtained by the (k+1)th transmission/reception, (k+3)th transmission/reception, ..., (k+9)th transmission/reception, and (k+11)th transmission/reception, five pieces of reflected wave data obtained by the (k+3)th transmission/reception, ..., (k+9)th transmission/reception, and (k+11)th transmission/reception are input to the eigenvector MTI filter. Similarly, although not illustrated in FIG. 11, among six pieces of reflected wave data obtained by the (k+2)th transmission/reception, (k+4)th transmission/reception, ..., (k+10)th transmission/reception, and (k+12)th transmission/reception, five pieces of reflected wave data obtained by the (k+4)th transmission/reception, ..., (k+10)th transmission/reception, and (k+12)th transmission/reception are input to the eigenvector MTI filter. In other words, at level 1, the packet size of the eigenvector MTI filter is five. This results in color Doppler image data corresponding to the first sub-region including the first scanning line n and the first scanning line n+1.

As illustrated in FIG. 11, at level 2, among six pieces of reflected wave data obtained by the (k+1)th transmission/reception, (k+3)th transmission/reception, ..., (k+9)th transmission/reception, and (k+11)th transmission/reception, four pieces of reflected wave data obtained by the (k+5)th transmission/reception, ..., (k+9)th transmission/reception, and (k+11)th transmission/reception are input to the eigenvector MTI filter. Similarly, although not illustrated in FIG. 11, among six pieces of reflected wave data obtained by the (k+2)th transmission/reception, (k+4)th transmission/reception, ..., (k+10)th transmission/reception, and (k+12)th transmission/reception, four pieces of reflected wave data obtained by the (k+6)th transmission/reception, ..., (k+10)th transmission/reception, and (k+12)th transmission/reception are input to the eigenvector MTI filter. In other words, at level 2, the packet size of the eigenvector MTI filter is four. This results in color Doppler image data corresponding to the first sub-region including the first scanning line n and the first scanning line n+1.

As illustrated in FIG. 11, at level 3, among six pieces of reflected wave data obtained by the (k+1)th transmission/reception, (k+3)th transmission/reception, ..., (k+9)th transmission/reception, and (k+11)th transmission/reception, three pieces of reflected wave data obtained by the (k+7)th transmission/reception, (k+9)th transmission/reception, and (k+11)th transmission/reception are input to the eigenvector MTI filter. Similarly, although not illustrated in FIG. 11, among six pieces of reflected wave data obtained by the (k+2)th transmission/reception, (k+4)th transmission/reception, ..., (k+10)th transmission/reception, and (k+12)th transmission/reception, three pieces of reflected wave data obtained by the (k+8)th transmission/reception, (k+10)th transmission/reception, and (k+12)th transmission/reception are input to the eigenvector MTI filter. In other words, at level 3, the packet size of the eigenvector MTI filter is three. This results in color Doppler image data corresponding to the first sub-region including the first scanning line n and the first scanning line n+1.

In the residual multiplex reduction mode, the higher the value of the level, the greater the degree of residual multiplex reduction. The lower the value of the level, the higher the image quality of color Doppler image data.

The image quality of color Doppler image data obtained in the dummy rate mode is higher than the image quality of color Doppler image data obtained in residual multiplex reduction mode. Therefore, if the operator has selected to emphasize the image quality of color Doppler image data (No at step S202), the ultrasonic diagnostic apparatus 1a sets the scanning mode to the dummy rate mode, alternately executes first ultrasonic scanning and second ultrasonic scanning in the dummy rate mode (see FIG. 4) for the subject P to generate B-mode image data and color Doppler image data (step S204), and then proceeds to step S105.

Here, at step S204, the processing of reducing residual multiplex at the level selected by the operator is performed. For example, as illustrated in FIG. 12, at level 1, the ultrasonic diagnostic apparatus 1a transmits an ultrasonic wave along the first scanning line n as the (k+1)th transmission/reception and transmits an ultrasonic wave along the first scanning line n+1 as the (k+2)th transmission/reception. However, the reception circuitry 112 of the ultrasonic diagnostic apparatus 1a does not generate reflected wave data from the reflected wave of the ultrasonic wave obtained in each of the (k+1)th transmission/reception and the (k+2)th transmission/reception. Alternatively, the circuitry on the subsequent stage to the reception circuitry 112 of the ultrasonic diagnostic apparatus 1a does not generate data using the reflected wave data generated from the reflected wave of the ultrasonic wave obtained in each of the (k+1)th transmission/reception and the (k+2)th transmission/reception.

The ultrasonic diagnostic apparatus 1a then repeats transmitting and receiving ultrasonic waves along the first scanning line n and transmitting and receiving ultrasonic waves along the first scanning line n+1, from the (k+3)th transmission/reception to the (k+14)th transmission/reception. In this way, the ultrasonic diagnostic apparatus 1a scans the first sub-region including the first scanning line n and the first scanning line n+1. This results in six pieces of reflected wave data corresponding to the first scanning line n and six pieces of reflected wave data corresponding to the first scanning line n+1.

The ultrasonic diagnostic apparatus 1a then inputs a data sequence including six pieces of reflected wave data corresponding to the first scanning line n to the eigenvector MTI filter to obtain color Doppler data. Similarly, the ultrasonic diagnostic apparatus 1a inputs a data sequence including six pieces of reflected wave data corresponding to the first scanning line n+1 to the eigenvector MTI filter to obtain color Doppler data. In other words, the packet size of the eigenvector MTI filter in the case of level 1 in the dummy rate mode is six. The ultrasonic diagnostic apparatus 1a then generates color Doppler image data corresponding to the first sub-region including the first scanning line n and the first scanning line n+1 from these pieces of obtained color Doppler data.

As illustrated in FIG. 12, at level 2, the ultrasonic diagnostic apparatus 1a transmits an ultrasonic wave along the first scanning line n as the (k+1)th transmission/reception, transmits an ultrasonic wave along the first scanning line n+1 as the (k+2)th transmission/reception, transmits an ultrasonic wave along the first scanning line n as the (k+3)th transmission/reception, and transmits an ultrasonic wave along the first scanning line n+1 as the (k+4)th transmission/reception. However, the reception circuitry 112 of the ultrasonic diagnostic apparatus 1a does not generate reflected wave data from the reflected wave of the ultrasonic wave obtained in each of the (k+1)th transmission/reception, (k+2)th transmission/reception, (k+3)th transmission/reception, and (k+4)th transmission/reception. Alternatively, the circuitry on the subsequent stage to the reception circuitry 112 of the ultrasonic diagnostic apparatus 1a does not generate data using the reflected wave data generated from the reflected wave of the ultrasonic wave obtained in each of the (k+1)th transmission/reception, (k+2)th transmission/reception, (k+3)th transmission/reception, and (k+4)th transmission/reception.

The ultrasonic diagnostic apparatus 1a then repeats transmitting and receiving ultrasonic waves along the first scanning line n and transmitting and receiving ultrasonic waves along the first scanning line n+1, from the (k+5)th transmission/reception to the (k+16)th transmission/reception. In this way, the ultrasonic diagnostic apparatus 1a scans the first sub-region including the first scanning line n and the first scanning line n+1. This results in six pieces of reflected wave data corresponding to the first scanning line n and six pieces of reflected wave data corresponding to the first scanning line n+1.

The ultrasonic diagnostic apparatus 1a then inputs a data sequence including six pieces of reflected wave data corresponding to the first scanning line n to the eigenvector MTI filter to obtain color Doppler data. Similarly, the ultrasonic diagnostic apparatus 1a inputs a data sequence including six pieces of reflected wave data corresponding to the first scanning line n+1 to the eigenvector MTI filter to obtain color Doppler data. In other words, the packet size of the eigenvector MTI filter in the case of level 2 in the dummy rate mode is six. The ultrasonic diagnostic apparatus 1a then generates color Doppler image data corresponding to the first sub-region including the first scanning line n and the first scanning line n+1 from these pieces of obtained color Doppler data.

As illustrated in FIG. 12, at level 3, the ultrasonic diagnostic apparatus 1a transmits an ultrasonic wave along the first scanning line n as the (k+1)th transmission/reception, transmits an ultrasonic wave along the first scanning line n+1 as the (k+2)th transmission/reception, transmits an ultrasonic wave along the first scanning line n as the (k+3)th transmission/reception, transmits an ultrasonic wave along the first scanning line n+1 as the (k+4)th transmission/reception, transmits an ultrasonic wave along the first scanning line n as the (k+5)th transmission/reception, and transmits an ultrasonic wave along the first scanning line n+1 as the (k+6)th transmission/reception. However, the reception circuitry 112 of the ultrasonic diagnostic apparatus 1a does not generate reflected wave data from the reflected wave of the ultrasonic wave obtained in each of the (k+1)th transmission/reception, (k+2)th transmission/reception, (k+3)th transmission/reception, (k+4)th transmission/reception, (k+5)th transmission/reception, and (k+6)th transmission/reception. Alternatively, the circuitry on the subsequent stage to the reception circuitry 112 of the ultrasonic diagnostic apparatus 1a does not generate data using the reflected wave data generated from the reflected wave of the ultrasonic wave obtained in each of the (k+1)th transmission/reception, (k+2)th transmission/reception, (k+3)th transmission/reception, (k+4)th transmission/reception, (k+5)th transmission/reception, and (k+6)th transmission/reception.

The ultrasonic diagnostic apparatus 1a then repeats transmitting and receiving ultrasonic waves along the first scanning line n and transmitting and receiving ultrasonic waves along the first scanning line n+1, from the (k+7)th transmission/reception to the (k+18)th transmission/reception. In this way, the ultrasonic diagnostic apparatus 1a scans the first sub-region including the first scanning line n and the first scanning line n+1. This results in six pieces of reflected wave data corresponding to the first scanning line n and six pieces of reflected wave data corresponding to the first scanning line n+1.

The ultrasonic diagnostic apparatus 1a then inputs a data sequence including six pieces of reflected wave data corresponding to the first scanning line n to the eigenvector MTI filter to obtain color Doppler data. Similarly, the ultrasonic diagnostic apparatus 1a inputs a data sequence including six pieces of reflected wave data corresponding to the first scanning line n+1 to the eigenvector MTI filter to obtain color Doppler data. In other words, the packet size of the eigenvector MTI filter in the case of level 3 in the dummy rate mode is six. The ultrasonic diagnostic apparatus 1a then generates color Doppler image data corresponding to the first sub-region including the first scanning line n and the first scanning line n+1 from these pieces of obtained color Doppler data.

In the dummy rate mode, the higher the value of the level, the greater the degree of residual multiplex reduction. The image quality of color Doppler image data is constant regardless of the value of the level.

The ultrasonic diagnostic apparatus 1a according to the second embodiment has been described above.

In the second embodiment, the scanning unit includes, for example, the ultrasound probe 101, the transmission/reception circuitry 110, and the control circuitry 180a, and is implemented by the ultrasound probe 101, the transmission/reception circuitry 110, and the control circuitry 180a. However, the scanning unit may further include circuitry and/or equipment other than these. In the second embodiment, the generation unit includes, for example, the transmission/reception circuitry 110, the B-mode processing circuitry 130, the Doppler processing circuitry 140, the image generation circuitry 150, and the control circuitry 180a, and is implemented by the transmission/reception circuitry 110, the B-mode processing circuitry 130, the Doppler processing circuitry 140, the image generation circuitry 150, and the control circuitry 180a. However, the generation unit may further include circuitry and/or equipment other than these.

In the second embodiment, when the first ultrasonic scanning is executed by the scanning unit after the second ultrasonic scanning in the dummy rate mode, the generation unit generates, as at least part of color Doppler image data representing the entire first region, color Doppler image data representing a region (one first sub-region) in the first region that corresponds to 12 transmissions / receptions, based on 12 reflected wave signals obtained by a fourth predetermined number of transmissions / receptions (12 transmissions / receptions in each of levels 1 to 3 in FIG. 12), which is greater than a second predetermined number, excluding a third predetermined number of transmissions / receptions from the first transmission/reception (two transmissions / receptions from the first transmission / reception (two transmissions / receptions indicated by the triangular frames) at level 1 in FIG. 12, four transmissions / receptions from the first transmission / reception (four transmissions / receptions indicated by the triangular frames) at level 2, and six transmissions / receptions from the first transmission / reception (six transmissions / receptions indicated by the triangular frames) at level 3), among all transmissions / receptions in the first ultrasonic scanning. The dummy rate mode is, for example, an example of a second scanning mode.

When the first ultrasonic scanning is executed by the scanning unit after the second ultrasonic scanning in the dummy rate mode, the generation unit generates, as at least part of color Doppler image data representing the entire first region, color Doppler image data representing the first sub-region corresponding to the fourth predetermined number of transmissions / receptions, using 12 reflected wave signals obtained by the fourth predetermined number of transmissions / receptions, and the eigenvector MTI filter.

In the second embodiment, the input device 102 accepts an instruction to emphasize the frame rate of color Doppler image data (frame rate emphasis instruction). The input device 102 also accepts specification of a level at which the residual multiplex in color Doppler image data is to be reduced (level specification). When the input device 102 accepts the frame rate emphasis instruction and the level specification, the generation unit changes the number of transmissions / receptions for a first predetermined number of transmissions / receptions and changes the number of transmissions / receptions for a second predetermined number of transmissions / receptions so that the total number of transmissions / receptions, which is the sum of the number of transmissions / receptions for the first predetermined number of transmissions / receptions ("1" for level 1, "2" for level 2, and "3" for level 3 in FIG. 11) and the number of transmissions / receptions for the second predetermined number of transmissions / receptions ("5" for level 1, "4" for level 2, and "3" for level 3 in FIG. 11), is constant at "6", in the residual multiplex reduction mode. The input device 102 is, for example, an example of an acceptance unit.

In the second embodiment, the input device 102 accepts an instruction to emphasize the image quality of color Doppler image data (image quality emphasis instruction). The input device 102 also accepts a level specification at which residual multiplex in color Doppler image data is to be reduced. Then, when the input device 102 accepts the image quality emphasis instruction and the level specification, the generation unit changes the number of transmissions / receptions for the third predetermined number of transmissions / receptions without changing the number of transmissions / receptions for the fourth predetermined number of transmissions / receptions, in the dummy rate mode.

In the second embodiment, the generation unit generates a color Doppler image using a piece or pieces of reflected wave data corresponding to a fourth number of pieces of reflected wave data, which is greater than a second number of pieces of reflected wave data, excluding a piece or pieces of reflected wave data corresponding to a third number of pieces of reflected wave data not to be used for color Doppler image generation, counted from the first transmission/reception, among a plurality of pieces of reflected wave data collected by multiple ultrasonic transmissions / receptions on a predetermined scanning line, in the dummy rate mode.

In the second embodiment, the generation unit generates a color Doppler image using a piece or pieces of reflected wave data corresponding to the fourth number of pieces of reflected wave data and the eigenvector MTI filter, in the dummy rate mode.

In the second embodiment, the input device 102 accepts a frame rate emphasis instruction to emphasize the frame rate of the color Doppler image and a level specification at which residual multiplex in the color Doppler image is to be reduced. When the input device 102 accepts the frame rate emphasis instruction and the level specification, the generation unit changes the first number of pieces of reflected wave data and changes the second number of pieces of reflected wave data so that the total number of pieces of reflected wave data, which is the sum of the first number of pieces of reflected wave data and the second number of pieces of reflected wave data, is constant, in the residual multiplex reduction mode.

In the second embodiment, the input device 102 accepts an image quality emphasis instruction to emphasize the image quality of the color Doppler image and a level specification at which residual multiplex in the color Doppler image is to be reduced. When the input device 102 accepts the image quality emphasis instruction and the level specification, the generation unit changes the third number of pieces of reflected wave data without changing the fourth number of pieces of reflected wave data, in the dummy rate mode.

In the second embodiment, in the processing at S203 and the processing at S204, the generation unit determines the number of pieces of reflected wave data not to be used for color Doppler image generation according to the effect of a reflected wave signal (echo signal) transmitted/received at a time phase prior to the multiple ultrasonic transmissions / receptions on the predetermined scanning line.

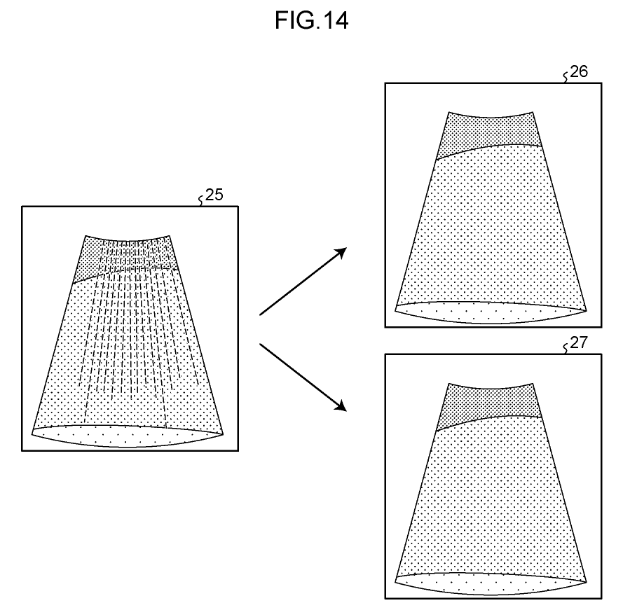

FIG. 14 is a diagram illustrating an example of a color Doppler image displayed by the ultrasonic diagnostic apparatus 1a according to the second embodiment. FIG. 14 illustrates a color Doppler image 25 displayed on the display 103 in the normal mode, a color Doppler image 26 displayed on the display 103 in the residual multiplex reduction mode, and a color Doppler image 27 displayed on the display 103 in the dummy rate mode.

Comparing the color Doppler image 25 with the color Doppler images 26 and 27, it can be understood that artifacts due to the effect of residual multiplex occur in the color Doppler image 20, while the occurrence of artifacts is suppressed in the color Doppler images 26 and 27. Comparing the color Doppler image 26 with the color Doppler image 27, the image quality of the color Doppler image 27 is better than the image quality of the color Doppler image 26. However, as described above, the frame rate of the color Doppler image 26 is higher than the frame rate of the color Doppler image 27.

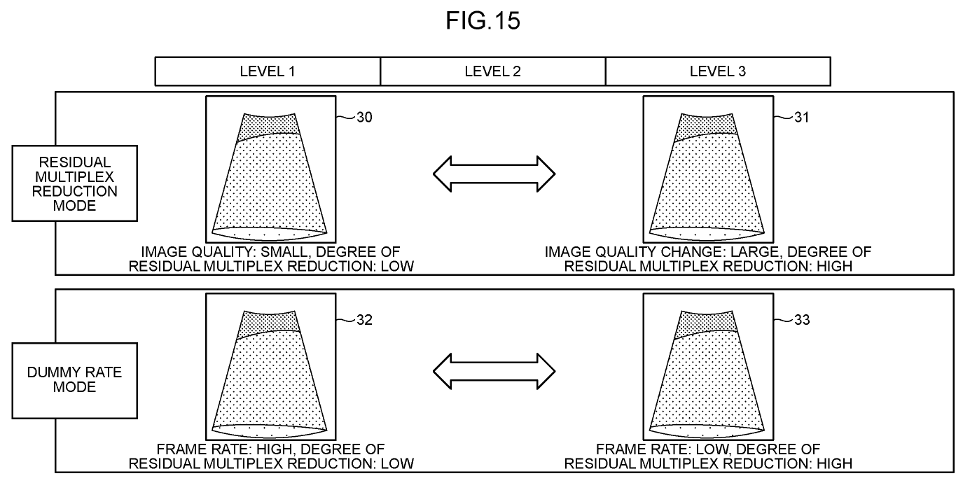

FIG. 15 is a diagram for explaining the features of color Doppler images displayed on the display 103 in the ultrasonic diagnostic apparatus 1a according to the second embodiment, for each combination of level and scanning mode. FIG. 15 illustrates a color Doppler image 30 based on color Doppler image data obtained at level 1 in the residual multiplex reduction mode, a color Doppler image 31 based on color Doppler image data obtained at level 3 in the residual multiplex reduction mode, a color Doppler image 32 based on color Doppler image data obtained at level 1 in the dummy rate mode, and a color Doppler image 33 based on color Doppler image data obtained at level 3 in the dummy rate mode.

Comparing the color Doppler image 30 with the color Doppler image 31, the change in the color Doppler image 31 is greater than the change in the color Doppler image 30 in terms of the change in image quality from the color Doppler image based on color Doppler image data obtained in the normal mode. The degree of residual multiplex reduction in the color Doppler image 31 is higher than the degree of residual multiplex reduction in the color Doppler image 30.

Comparing the color Doppler image 32 with the color Doppler image 33, the frame rate of the color Doppler image 32 is higher than the frame rate of the color Doppler image 33. The degree of residual multiplex reduction in the color Doppler image 33 is higher than the degree of residual multiplex reduction in the color Doppler image 32.

Based on the above, the ultrasonic diagnostic apparatus 1a according to the second embodiment can suppress the reduction in the frame rate or the image quality of the color Doppler image as emphasized by the operator. The ultrasonic diagnostic apparatus 1a according to the second embodiment can reduce residual multiplex at the level desired by the operator.

### Third Embodiment

An ultrasonic diagnostic apparatus according to a third embodiment will now be described. In the description of the third embodiment, the same configuration as in the first and second embodiments is denoted by the same reference sign and the description thereof may be omitted. In the description of the third embodiment, the points different from the first and second embodiments will be mainly described.

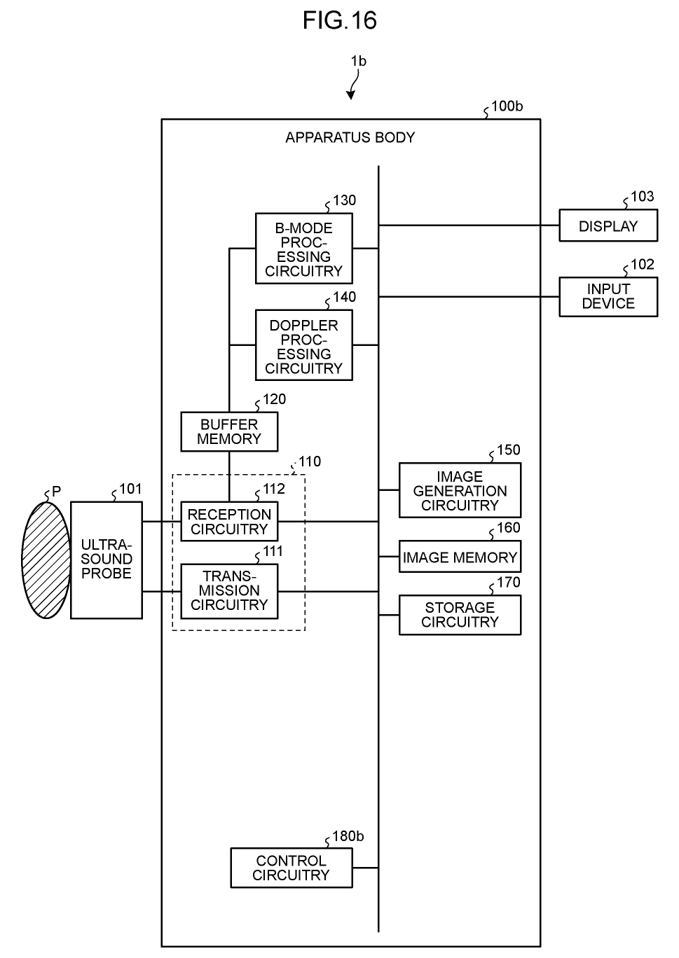

FIG. 16 is a block diagram illustrating an example configuration of an ultrasonic diagnostic apparatus 1b according to the third embodiment. The ultrasonic diagnostic apparatus 1b according to the third embodiment differs from the ultrasonic diagnostic apparatus 1, 1a in that it includes an apparatus body 100b instead of the apparatus body 100, 100a. The apparatus body 100b according to the third embodiment differs from the apparatus body 100, 100a in that it includes control circuitry 180b instead of the control circuitry 180, 180a.

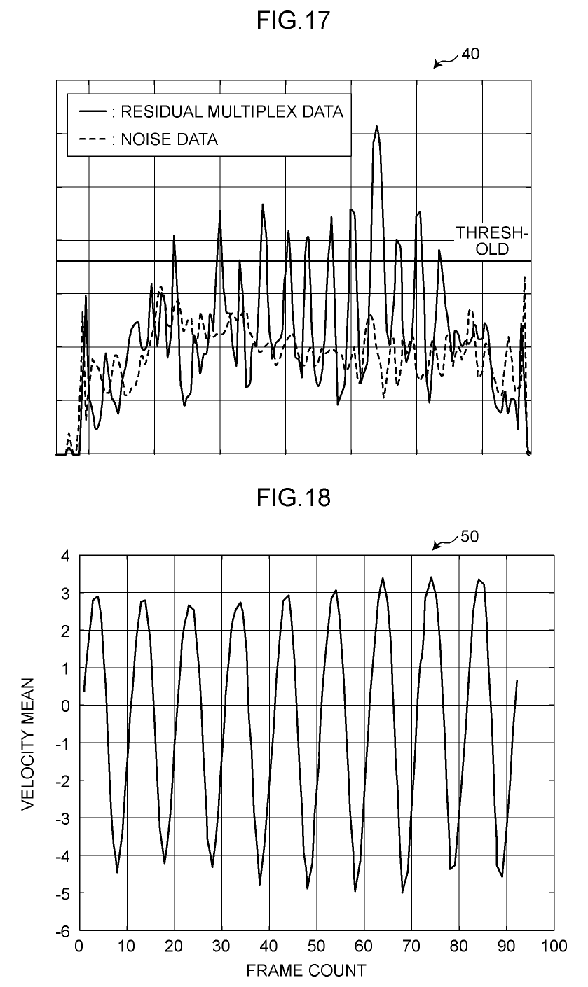

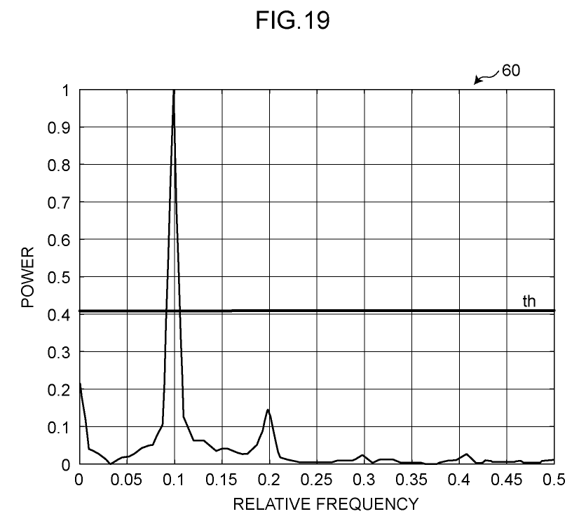

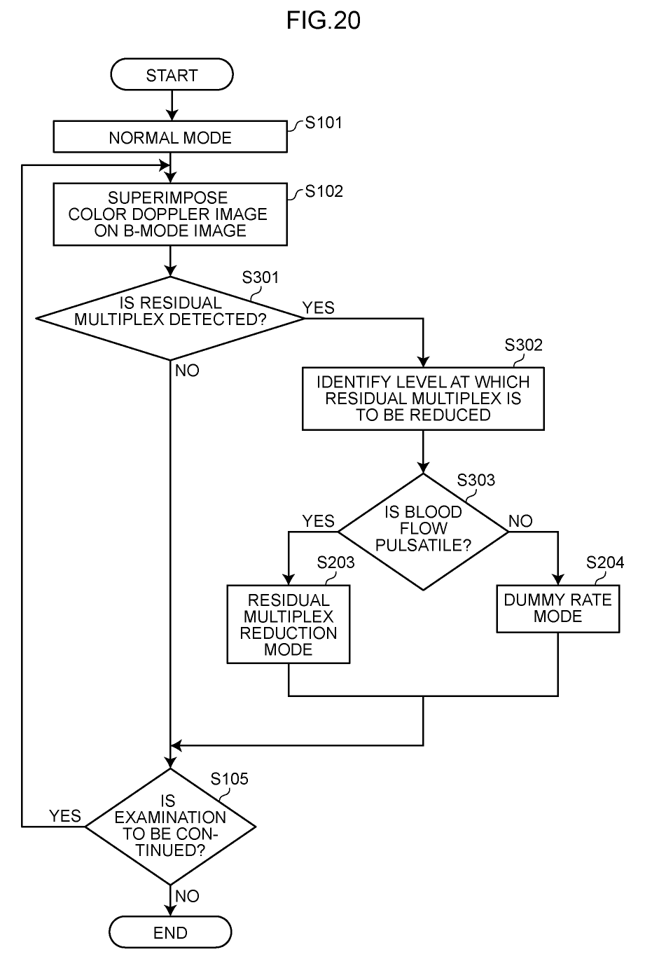

FIG. 17 is a diagram for explaining an example of processing for detecting residual multiplex in the third embodiment. FIG. 18 is a diagram for explaining an example of processing for detecting whether there is pulsatility in the third embodiment. FIG. 19 is a diagram for explaining an example of processing for detecting whether there is pulsatility in the third embodiment. FIG. 20 is a flowchart illustrating the flow of exemplary processing executed by the ultrasonic diagnostic apparatus 1b according to the third embodiment.

The processing illustrated in FIG. 20 is executed when the operator operates the input device 102 and inputs an instruction to the control circuitry 180b to superimpose a color Doppler image on a B-mode image for the examination of the subject P.

The processing at each of steps S101, S102, S203, S204, and S105 illustrated in FIG. 20 is similar to the processing at each of steps S101, S102, S203, S204, and S105 illustrated in FIG. 13. However, the processing executed by the control circuitry 180a in FIG. 13 is executed by the control circuitry 180b in FIG. 20.

As illustrated in FIG. 20, the control circuitry 180b determines whether residual multiplex has occurred in a color Doppler image (step S301). For example, the control circuitry 180b attempts to detect residual multiplex in the color Doppler image data, and if residual multiplex is detected, the control circuitry 180b determines that residual multiplex has occurred in the color Doppler image (Yes at step S301) and then proceeds to step S302. On the other hand, if no residual multiplex is detected, the control circuitry 180b determines that no residual multiplex occurs in the color Doppler image (No at step S301) and then proceeds to step S105.

An example of the determination processing at step S301 will be described. When residual multiplex occurs in color Doppler image data, the color Doppler image data has first to fourth features described below. The first feature is that the velocity of blood flow indicated by color Doppler image data is not constant but unstable. The second feature is that the power of blood flow indicated by color Doppler image data is relatively high. The third feature is that residual multiplex enters the first scanning line (the scanning line on which ultrasonic waves are transmitted and received first) of an alternating stage group. The fourth feature is that the continuity of residual multiplex is high in the depth direction.

The control circuitry 180b, for example, attempts to detect residual multiplex from color Doppler image data as follows, based on the second and third features among the first to fourth features. For example, as illustrated in FIG. 17, the control circuitry 180b generates a graph 40 indicating the power value of residual multiplex and the power value of noise for each first scanning line, from the color Doppler image data. The horizontal axis of graph 40 indicates the first scanning lines, and the vertical axis indicates the power values. The control circuitry 180b then determines, from the graph 40, whether the first scanning line with a power value of residual multiplex equal to or greater than a threshold (see FIG. 17) exists periodically (for each alternating stage group) repeatedly in the scanning line direction. In the example of FIG. 17, the control circuitry 180b determines that the first scanning line with a power value of residual multiplex equal to or greater than the threshold exists periodically repeatedly in the scanning line direction. If it is determined that the first scanning line with a power value of residual multiplex equal to or greater than the threshold exists periodically repeatedly in the scanning line direction, the control circuitry 180b determines that residual multiplex is detected in the color Doppler image data (Yes at step S301). On the other hand, if it is determined that the first scanning line with a power value of residual multiplex equal to or greater than the threshold does not exist periodically repeatedly in the scanning line direction, the control circuitry 180b determines that residual multiplex is not detected from the color Doppler image data (No at step S301).

The control circuitry 180b then automatically identifies the degree (level) at which residual multiplex is to be reduced (step S302).

An example of the processing at step S302 will be described. The control circuitry 180b identifies a level at which residual multiplex is to be reduced as follows. For example, each time the control circuitry 180b determines to continue the examination at step S105 (Yes at step S105), the control circuitry 180b determines whether residual multiplex has occurred in the color Doppler image, at step S301. Therefore, if it is repeatedly determined to continue the examination at step S105 (Yes at step S105), it is repeatedly determined whether residual multiplex has occurred in the color Doppler image, at step S301. Therefore, if it is determined that residual multiplex has occurred in the color Doppler image a predetermined number of times in succession, at step S301 (Yes at step S301), the control circuitry 180b automatically identifies the level by increasing the level at which residual multiplex is to be reduced by one.

The control circuitry 180b then determines whether the blood flow is pulsatile (step S303). For example, the image memory 160 stores therein a plurality of pieces of color Doppler image data (color Doppler image data for a plurality of frames) obtained in the normal mode. The control circuitry 180b then uses these pieces of color Doppler image data to determine whether the blood flow is pulsatile. For example, the control circuitry 180b calculates the average velocity of blood flow within a frame for each frame. The control circuitry 180b then generates the graph 50 indicating the average velocity of blood flow for each frame, as illustrated in FIG. 18. The horizontal axis of the graph 50 indicates the frames, and the vertical axis indicates the average velocity of blood flow. The graph 50 indicates the frame-to-frame change of the average velocity of blood flow. The graph 50 also indicates the heart rate of the subject P.

The control circuitry 180b then performs frequency analysis on the graph 50 to acquire a normalized power value for each frequency. The control circuitry 180b thereby acquires the graph 60 indicating the normalized power value for each frequency, as illustrated in FIG. 19. The horizontal axis of the graph 60 indicates the frequencies, and the vertical axis indicates the power values normalized to a range from 0 to 1.

The control circuitry 180b then determines from the graph 60 that the blood flow is pulsatile (Yes at step S303) if there is a power value exceeding a threshold th (see FIG. 19) among the power values at frequencies other than direct current (frequencies other than 0 Hz). On the other hand, the control circuitry 180b determines from the graph 60 that the blood flow is not pulsatile (No at step S303) if there is no power value exceeding the threshold th among the power values at frequencies other than direct current.

Here, if the blood flow is pulsatile, time resolution is important and real-time performance is required. Therefore, if the blood flow is pulsatile (Yes at step S303), the ultrasonic diagnostic apparatus 1b sets the scanning mode to the residual multiplex reduction mode, alternately executes first ultrasonic scanning and second ultrasonic scanning in the residual multiplex reduction mode for the subject P to generate B-mode image data and color Doppler image data (step S203), and proceeds to step S105.

If the blood flow is not pulsatile, the blood flow is flowing at a nearly constant rate, so time resolution is less important and real-time performance is less required. Therefore, if the blood flow is not pulsatile (No at step S303), the ultrasonic diagnostic apparatus 1b sets the scanning mode to the dummy rate mode, alternately executes first ultrasonic scanning and second ultrasonic scanning in the dummy rate mode for the subject P to generate B-mode image data and color Doppler image data (step S204), and proceeds to step S105.

Here, in each of steps S203 and S204, the ultrasonic diagnostic apparatus 1b performs the processing of reducing residual multiplex at the level identified at step S302, in the same way as in the second embodiment.

The ultrasonic diagnostic apparatus 1b may include an electrocardiograph that acquires electrocardiogram (ECG) as a biological signal of the subject P. The control circuitry 180b may then perform the processing at step S303 using the electrocardiogram acquired by the electrocardiograph. For example, the control circuitry 180b compares the heart rate indicated by the graph 50 with the heart rate indicated by the electrocardiogram, and calculates the degree of agreement indicating the degree to which the heart rate indicated by the graph 50 agrees with the heart rate indicated by the electrocardiogram. The higher the value indicated by the degree of agreement, the higher the degree of agreement to which the heart rate indicated by the graph 50 agrees with the heart rate indicated by the electrocardiogram. If the degree of agreement exceeds a predetermined threshold, the graph 50 is considered as indicating the actual heart rate of the subject P. The control circuitry 180b therefore performs the processing at step S303 above using the graph 50. If the degree of agreement is equal to or smaller than a predetermined threshold, the graph 50 is considered as not indicating the actual heart rate of the subject P. The control circuitry 180b therefore newly generates the graph 50 indicating the average velocity of blood flow for each frame, using another plurality of pieces of color Doppler image data stored in the image memory 160. The control circuitry 180b then performs the processing described above using the newly generated graph 50. The control circuitry 180b repeatedly executes such processing until the degree of agreement exceeds the predetermined threshold. The electrocardiograph is, for example, an example of an acquisition unit.

If the degree of agreement is equal to or smaller than the predetermined threshold, the control circuitry 180b may determine that the blood flow is not pulsatile (No at step S303), rather than newly generating the graph 50, and may proceed to step S204.

Although the case where the control circuitry 180b performs the processing of determining whether the blood flow is pulsatile at step S303 has been described, different processing may be performed at step S303. For example, at step S303, the control circuitry 180b identifies at least one pair of two adjacent frames (a pair of color Doppler image data) in the frame direction of the color Doppler image data. The control circuitry 180b then correlates the two paired frames, for each pair. In other words, the control circuitry 180b calculates a correlation value of two pieces of color Doppler image data, for each pair.

If one pair is identified, the control circuitry 180b determines whether the calculated correlation value is equal to or smaller than a predetermined threshold. If the correlation value is equal to or smaller than the predetermined threshold (Yes at step S303), the control circuitry 180b proceeds to step S203. On the other hand, if the correlation value exceeds the predetermined threshold (No at step S303), the control circuitry 180b proceeds to step S204.

If a plurality of pairs are identified, the control circuitry 180b calculates the average of a plurality of the calculated correlation values. The control circuitry 180b then determines whether the average of a plurality of correlation values is equal to or smaller than a predetermined threshold. If the average of a plurality of correlation values is equal to or smaller than the predetermined threshold (Yes at step S303), the control circuitry 180b proceeds to step S203. On the other hand, if the average of a plurality of correlation values exceeds the predetermined threshold (No at step S303), the control circuitry 180b proceeds to step S204.

The ultrasonic diagnostic apparatus 1b according to the third embodiment has been described above.

In the third embodiment, the scanning unit includes, for example, the ultrasound probe 101, the transmission/reception circuitry 110, and the control circuitry 180b, and is implemented by the ultrasound probe 101, the transmission/reception circuitry 110, and the control circuitry 180b. However, the scanning unit may further include circuitry and/or equipment other than these. In the third embodiment, the generation unit includes, for example, the transmission/reception circuitry 110, the B-mode processing circuitry 130, the Doppler processing circuitry 140, the image generation circuitry 150, and the control circuitry 180b, and is implemented by the transmission/reception circuitry 110, the B-mode processing circuitry 130, the Doppler processing circuitry 140, the image generation circuitry 150, and the control circuitry 180b. However, the generation unit may further include circuitry and/or equipment other than these.

In the third embodiment, the generation unit generates a plurality of pieces of color Doppler image data, determines whether the blood flow is pulsatile based on the velocity of blood flow indicated by the pieces of color Doppler image data, and when it is determined that the blood flow is pulsatile, generates, as at least part of color Doppler image data representing the first region, color Doppler image data representing the first sub-region corresponding to a second predetermined number of transmissions/receptions in the residual multiplex reduction mode.

In the third embodiment, when it is determined that the blood flow is not pulsatile, the generation unit generates, as at least part of color Doppler image data representing the first region, color Doppler image data representing the first sub-region corresponding to a fourth predetermined number of transmissions/receptions in the dummy rate mode.

In the third embodiment, the electrocardiograph acquires an electrocardiogram as a biological signal of the subject P. The generation unit then determines whether the blood flow is pulsatile, based on the velocity of blood flow indicated by a plurality of pieces of color Doppler image data and the electrocardiogram.

In the third embodiment, each time color Doppler image data is newly generated, the generation unit determines whether residual multiplex has occurred in the newly generated color Doppler image data. When it is determined that residual multiplex has occurred a predetermined number of times in succession, the generation unit identifies a level at which the residual multiplex in the color Doppler image data is to be reduced. When it is determined that the blood flow is pulsatile, the generation unit changes the number of transmissions/receptions for a first predetermined number of transmissions/receptions and changes the number of transmissions/receptions for a second predetermined number of transmissions/receptions so that the total number of transmissions/receptions, which is the sum of the number of transmissions/receptions for the first predetermined number of transmissions/receptions ("1" for level 1, "2" for level 2, and "3" for level 3 in FIG. 11) and the number of transmissions/receptions for the second predetermined number of transmissions/receptions ("5" for level 1, "4" for level 2, and "3" for level 3 in FIG. 11) is constant at "6", based on the identified level, in the residual multiplex reduction mode.

In the third embodiment, if it is determined that the blood flow is not pulsatile, the generation unit changes the number of transmissions/receptions for the third predetermined number of transmissions/receptions, without changing the number of transmissions/receptions for the fourth predetermined number of transmissions/receptions, based on the identified level, in the dummy rate mode.

In the third embodiment, the generation unit generates a plurality of pieces of color Doppler image data, and generates, as at least part of color Doppler image data representing the first region, color Doppler image data representing the first sub-region corresponding to a second predetermined number of transmissions/receptions in the residual multiplex reduction mode, or generates, as at least part of color Doppler image data representing the first region, color Doppler image data representing the first sub-region corresponding to a fourth predetermined number of transmissions/receptions in the dummy rate mode, based on a correlation value of at least one pair of two pieces of color Doppler image data adjacent to each other in the frame direction among the pieces of color Doppler image data.

In the third embodiment, the generation unit generates a plurality of color Doppler images, determines whether the blood flow is pulsatile based on the velocity of blood flow indicated by the color Doppler images, and when it is determined that the blood flow is pulsatile, sets the scanning mode of the ultrasonic diagnostic apparatus 1b to the residual multiplex reduction mode.

In the third embodiment, the generation unit generates a plurality of color Doppler images, determines whether the blood flow is pulsatile based on the velocity of blood flow indicated by the color Doppler images, and when it is determined that the blood flow is not pulsatile, sets the scanning mode of the ultrasonic diagnostic apparatus 1b to the dummy rate mode.

In the third embodiment, the generation unit determines whether the blood flow is pulsatile, based on the velocity of blood flow indicated by a plurality of color Doppler images and an electrocardiogram of the subject P acquired by the electrocardiograph.

In the third embodiment, each time a color Doppler image is newly generated, the generation unit determines whether residual multiplex has occurred in the newly generated color Doppler image. When it is determined that residual multiplex has occurred a predetermined number of times in succession, the generation unit identifies a level at which residual multiplex in the color Doppler image is to be reduced. When it is determined that the blood flow is pulsatile, the generation unit changes the first number of pieces of reflected wave data and changes the second number of pieces of reflected wave data so that the total number of pieces of reflected wave data, which is the sum of the first number of pieces of reflected wave data and the second number of pieces of reflected wave data, is constant, in the residual multiplex reduction, based on the identified level.

In the third embodiment, each time a color Doppler image is newly generated, the generation unit determines whether residual multiplex has occurred in the newly generated color Doppler image. When it is determined that residual multiplex has occurred a predetermined number of times in succession, the generation unit identifies a level at which residual multiplex in the color Doppler image is to be reduced. When it is determined that the blood flow is not pulsatile, the generation unit changes a third number of pieces of reflected wave data without changing a fourth number of pieces of reflected wave data, in the dummy rate mode, based on the identified level.

In the third embodiment, the generation unit generates a plurality of color Doppler images and sets the scanning mode of the ultrasonic diagnostic apparatus 1b to the residual multiplex reduction mode or the dummy rate mode, based on a correlation value of at least one pair of two color Doppler images adjacent to each other in the frame direction among the color Doppler images.

The first to third embodiments describe a case where transmission/reception for the first scanning line n and the first scanning line n+1 (first ultrasonic scanning for obtaining color Doppler image data) is performed during transmission/reception for the second scanning line m and the second scanning line m+1 (second ultrasonic scanning for obtaining B-mode image data), and the color Doppler image data is affected by the second ultrasonic scanning and the residual signal may cause artifacts in a color Doppler image. However, situations in which the residual signal may cause artifacts in a color Doppler image are not limited to this.

For example, the following describes a case where the transmission/reception circuitry 110 performs a mode division scan (mode division scanning) in which first ultrasonic scanning for the entire first region and second ultrasonic scanning for the entire second region are alternately executed for the subject P through the ultrasound probe 101. The second ultrasonic scanning in the mode division scan is scanning in which one ultrasonic transmission/reception is performed for each of all second scanning lines (M second scanning lines) in the second region.

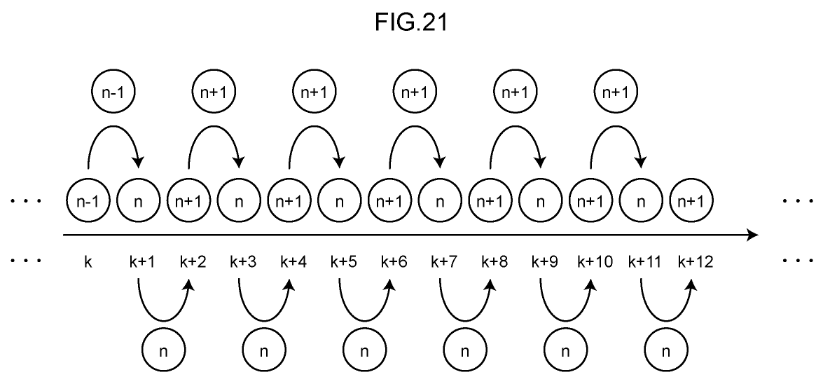

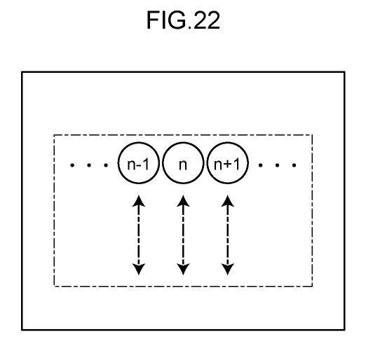

FIG. 21 is a diagram for explaining an example of the first ultrasonic scanning in the mode division scan. FIG. 22 is a diagram illustrating an example of the positions of a plurality of scanning lines (positions in spatial coordinates of image data) in the first ultrasonic scanning in the mode division scan. For example, as illustrated in FIGS. 21 and 22, the first ultrasonic scanning in the mode division scan is scanning in which multiple ultrasonic transmissions/receptions are performed for each of N scanning lines by continuously performing ultrasonic transmission/reception multiple times for one alternating stage group while changing the alternating stage groups, where the number of alternating stages is two. Specifically, in the example in FIG. 21, the scanning unit or the collection unit described above performs ultrasonic transmission/reception six times for an alternating stage group (first alternating stage group) of two first scanning lines n-2 (not illustrated) and n-1 on which ultrasonic waves are transmitted and received alternately. This results in six pieces of reflected wave data corresponding to the first scanning line n-2 and six pieces of reflected wave data corresponding to the first scanning line n-1. The scanning unit or the collection unit thereafter performs ultrasonic transmission/reception six times for an alternating stage group (second alternating stage group) of two first scanning lines n and n+1 on which ultrasonic waves are transmitted and received alternately. This results in six pieces of reflected wave data corresponding to the first scanning line n and six pieces of reflected wave data corresponding to the first scanning line n+1.

Here, in the first ultrasonic scanning in the mode division scan, the residual signal may cause artifacts in a color Doppler image at the border of the alternating stage group. For example, the residual signal of the ultrasonic wave transmitted last to the first alternating stage group (ultrasonic wave transmitted along the first scanning line n-1 as the kth transmission/reception) may enter the reception period of the reflected wave of the ultrasonic wave transmitted first to the second alternating stage group (ultrasonic wave transmitted along the first scanning line n as the (k+1)th transmission/reception). Therefore, the reflected wave signal on the first scanning line n obtained by the (k+1)th transmission/reception output from the ultrasound probe 101 contains the residual signal and therefore is affected by the residual signal.

Further, the residual signals of ultrasonic waves transmitted along the first scanning line n+1 as the (k+2)th transmission/reception, (k+4)th transmission/reception, (k+6)th transmission/reception, ..., and (k+10)th transmission/reception enter the reception periods of the reflected waves of the ultrasonic waves transmitted along the first scanning line n as the (k+3)th transmission/reception, (k+5)th transmission/reception, (k+7)th transmission/reception, ..., and (k+11)th transmission/reception, respectively. However, the residual signals contained in the reflected wave signal on the first scanning line n obtained by the (k+3)th transmission/reception, the reflected wave signal on the first scanning line n obtained by the (k+5)th transmission/reception, the reflected wave signal on the first scanning line n obtained by the (k+7)th transmission/reception, ..., and the reflected wave signal on the first scanning line n obtained by the (k+11)th transmission/reception, output from the ultrasound probe 101, are similar signals because the transmission/reception that is a source of the residual signals is based on the same ultrasonic transmission/reception conditions (ultrasonic transmission/reception conditions for the second alternating stage group for collecting color Doppler image data). Therefore, the residual signals contained in the reflected wave signals on the first scanning line n obtained by the (k+3)th transmission/reception, (k+5)th transmission/reception, (k+7)th transmission/reception, ..., and (k+11)th transmission/reception are unlikely to cause artifacts in a color Doppler image.

On the other hand, the residual signal contained in the reflected wave signal on the first scanning line n obtained by the (k+1)th transmission/reception output from the ultrasound probe 101 is a signal generated not by the transmission/reception based on the ultrasonic transmission/reception conditions for the second alternating stage group for collecting color Doppler image data but by the transmission/reception based on the ultrasonic transmission/reception conditions for the first alternating stage group for collecting color Doppler image data. Therefore, the aspect of the residual signal contained in the reflected wave signal on the first scanning line n obtained by the (k+1)th transmission/reception is significantly different from the aspect of the residual signals contained in the reflected wave signals on the first scanning line n obtained by the (k+3)th transmission/reception, (k+5)th transmission/reception, (k+7)th transmission/reception, ..., and (k+11)th transmission/reception. Therefore, the residual signal contained in the reflected wave signal on the first scanning line n obtained by the (k+1)th transmission/reception is imaged as an artifact image and becomes a cause of artifacts in the color Doppler image.

Note that the residual signals of ultrasonic waves transmitted along the first scanning line n as the (k+1)th transmission/reception, (k+3)th transmission/reception, (k+5)th transmission/reception, ..., and (k+11)th transmission/reception enter the reception periods of the reflected waves of the ultrasonic waves transmitted along the first scanning line n+1 as the (k+2)th transmission/reception, (k+4)th transmission/reception, (k+6)th transmission/reception, ..., and (k+12)th transmission/reception, respectively. However, the residual signals contained in the reflected wave signal on the first scanning line n+1 obtained by the (k+2)th transmission/reception, the reflected wave signal on the first scanning line n+1 obtained by the (k+4)th transmission/reception, the reflected wave signal on the first scanning line n+1 obtained by the (k+6)th transmission/reception, ..., and the reflected wave signal on the first scanning line n+1 obtained by the (k+12)th transmission/reception, output from the ultrasound probe 101, are similar signals because the transmission/reception that is a source of the residual signals is based on the same ultrasonic transmission/reception conditions (ultrasonic transmission/reception conditions for the second alternating stage group for collecting color Doppler image data). Therefore, the residual signals contained in the reflected wave signals on the first scanning line n+1 obtained by the (k+2)th transmission/reception, (k+4)th transmission/reception, (k+6)th transmission/reception, ..., and (k+12)th transmission/reception are unlikely to cause artifacts in a color Doppler image.

As described above, the residual signal contained in the reflected wave signal on the first scanning line n obtained by the (k+1)th transmission/reception is imaged as an artifact image and becomes a cause of artifacts in the color Doppler image. The ultrasonic diagnostic apparatus 1, 1a, 1b then may reduce the residual signal contained in the reflected wave signal on the first scanning line n obtained by the (k+1)th transmission/reception, using the same method as the method described above for reducing the residual signal contained in the reflected wave signal.

The computer program to be executed by the processor is embedded in advance and provided in a read only memory (ROM), storage circuitry, or the like. The computer program may be recorded and provided as a file in a format that can be installed on these devices or in an executable format on a non-transitory computer-readable medium such as a compact disc (CD)-ROM, a flexible disk (FD), a CD-R (recordable), and a digital versatile disc (DVD). The computer program may be provided or distributed by being stored on a computer connected to a network such as the Internet and downloaded over the network. For example, the computer program includes modules including each of the processing functions described above. As actual hardware, the CPU reads and executes a computer program from a recording medium such as a ROM, and each module is loaded onto the main memory and generated on the main memory.

According to at least one embodiment described above, the effect of residual multiplex on color Doppler image data can be reduced while suppressing the reduction in the frame rate of color Doppler image data.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus configured to generate a color Doppler image using a plurality of pieces of transmission / reception data obtained by performing ultrasonic transmission / reception multiple times on a same scanning line, the ultrasonic diagnostic apparatus comprising processing circuitry configured to:
collect a plurality of pieces of transmission/reception data by multiple ultrasonic transmissions / receptions on a predetermined scanning line; and
determine a number of pieces of transmission / reception data not to be used for color Doppler image generation according to an effect of an echo signal transmitted / received at a time phase prior to the multiple ultrasonic transmissions / receptions on the predetermined scanning line, and generate a color Doppler image using a plurality of pieces of transmission/reception data, excluding a piece or pieces of transmission / reception data corresponding to the determined number of pieces of transmission/reception data counted from a first piece of transmission / reception data, among the collected pieces of transmission/reception data.

2. The ultrasonic diagnostic apparatus according to claim 1, wherein
in a first scanning mode, the processing circuitry generates a color Doppler image using a piece or pieces of transmission/reception data corresponding to a second number of pieces of transmission/reception data, excluding a piece or pieces of transmission/reception data corresponding to a first number of pieces of transmission / reception data as the number of pieces of transmission / reception data not to be used for color Doppler image generation counted from a first piece of transmission/reception data, among the pieces of transmission / reception data collected by the multiple transmissions / receptions on the predetermined scanning line, the second number of pieces of transmission / reception data being equal to or smaller than a predetermined reference value, and
in a second scanning mode, the processing circuitry generates a color Doppler image using a piece or pieces of transmission / reception data corresponding to a fourth number of pieces of transmission / reception data, excluding a piece or pieces of transmission / reception data corresponding to a third number of pieces of transmission / reception data not to be used for color Doppler image generation counted from first transmission/reception, among the pieces of transmission / reception data collected by multiple ultrasonic transmissions / receptions on the predetermined scanning line, the fourth number of pieces of transmission / reception data being greater than the second number of pieces of transmission/reception data.

3. The ultrasonic diagnostic apparatus according to claim 2, wherein the processing circuitry generates a color Doppler image using a piece or pieces of transmission / reception data corresponding to the second number of pieces of transmission/reception data and an eigenvector MTI filter in the first scanning mode.

4. The ultrasonic diagnostic apparatus according to claim 2, wherein the processing circuitry generates a color Doppler image using a piece or pieces of transmission/reception data corresponding to the fourth number of pieces of transmission/reception data and an eigenvector MTI filter in the second scanning mode.

5. The ultrasonic diagnostic apparatus according to claim 2, further comprising an input interface configured to accept an instruction to emphasize a frame rate of the color Doppler image and specification of a level at which residual multiplex in the color Doppler image is to be reduced, wherein
when the input interface accepts the instruction and the specification, the processing circuitry changes the first number of pieces of transmission/reception data and changes the second number of pieces of transmission/reception data so that a total number of pieces of transmission/reception data is constant in the first scanning mode, the total number of pieces of transmission/reception data being a sum of the first number of pieces of transmission / reception data and the second number of pieces of transmission/reception data.

6. The ultrasonic diagnostic apparatus according to claim 2, further comprising an input interface configured to accept an instruction to emphasize an image quality of the color Doppler image and specification of a level at which residual multiplex in the color Doppler image is to be reduced, wherein
when the input interface accepts the instruction and the specification, the processing circuitry changes the third number of pieces of transmission/reception data without changing the fourth number of pieces of transmission/reception data in the second scanning mode.

7. The ultrasonic diagnostic apparatus according to claim 2, wherein the processing circuitry generates a plurality of the color Doppler images, determines whether blood flow is pulsatile based on a velocity of blood flow indicated by the color Doppler images, and when determining that blood flow is pulsatile, sets a scanning mode of the ultrasonic diagnostic apparatus to the first scanning mode.

8. The ultrasonic diagnostic apparatus according to claim 2, wherein the processing circuitry generates a plurality of the color Doppler images, determines whether blood flow is pulsatile based on a velocity of blood flow indicated by the color Doppler images, and when determining that blood flow is not pulsatile, sets a scanning mode of the ultrasonic diagnostic apparatus to the second scanning mode.

9. The ultrasonic diagnostic apparatus according to claim 7, wherein the processing circuitry determines whether blood flow is pulsatile, based on a velocity of blood flow indicated by the color Doppler images and a biological signal of a subject.

10. The ultrasonic diagnostic apparatus according to claim 8, wherein the processing circuitry determines whether blood flow is pulsatile, based on a velocity of blood flow indicated by the color Doppler images and a biological signal of a subject.

11. The ultrasonic diagnostic apparatus according to claim 7, wherein
each time the color Doppler image is newly generated, the generation unit determines whether residual multiplex occurs in the newly generated color Doppler image,
when determining that residual multiplex occurs a plurality of times in succession, the processing circuitry identifies a level at which residual multiplex in the color Doppler image is to be reduced, and
when determining that blood flow is pulsatile, the processing circuitry changes the first number of pieces of transmission/reception data and changes the second number of pieces of transmission/reception data so that a total number of pieces of transmission / reception data is constant, in the first scanning mode, based on the identified level, the total number of pieces of transmission / reception data being a sum of the first number of pieces of transmission/reception data and the second number of pieces of transmission/reception data.

12. The ultrasonic diagnostic apparatus according to claim 8, wherein
each time the color Doppler image is newly generated, the processing circuitry determines whether residual multiplex occurs in the newly generated color Doppler image,
when determining that residual multiplex occurs a plurality of times in succession, the processing circuitry identifies a level at which residual multiplex in the color Doppler image is to be reduced, and
when determining that blood flow is not pulsatile, the processing circuitry changes the third number of pieces of transmission / reception data without changing the fourth number of pieces of transmission/reception data, in the second scanning mode, based on the identified level.

13. The ultrasonic diagnostic apparatus according to claim 2, wherein the processing circuitry generates a plurality of the color Doppler images and sets a scanning mode of the ultrasonic diagnostic apparatus to the first scanning mode or the second scanning mode, based on a correlation value of at least one pair of two color Doppler images adjacent to each other in a frame direction among the color Doppler images.

14. A method for generating a color Doppler image using a plurality of pieces of transmission / reception data obtained by performing ultrasonic transmission / reception multiple times on a same scanning line, the method comprising:
collecting a plurality of pieces of transmission / reception data by multiple ultrasonic transmissions / receptions on a predetermined scanning line;
determining a number of pieces of transmission / reception data not to be used for color Doppler image generation according to an effect of an echo signal transmitted / received at a time phase prior to the multiple ultrasonic transmissions / receptions on the predetermined scanning line; and
generating a color Doppler image using a plurality of pieces of transmission / reception data, excluding a piece or pieces of transmission/reception data corresponding to the determined number of pieces of transmission / reception data counted from a first piece of transmission / reception data, among the collected pieces of transmission / reception data.

15. A non-transitory computer readable medium comprising instructions that cause a computer to execute processing for generating a color Doppler image using a plurality of pieces of transmission/reception data obtained by performing ultrasonic transmission / reception multiple times on a same scanning line, the processing comprising:
determining a number of pieces of transmission / reception data not to be used for color Doppler image generation according to an effect of an echo signal transmitted / received at a time phase prior to multiple ultrasonic transmissions / receptions on a predetermined scanning line; and
generating a color Doppler image using a plurality of pieces of transmission / reception data, excluding a piece or pieces of transmission/reception data corresponding to the determined number of pieces of transmission/reception data counted from a first piece of transmission / reception data, among the pieces of transmission/reception data obtained by the multiple ultrasonic transmissions / receptions on the predetermined scanning line.
